SEQUENCE LISTING (101 sequences)

SEQ ID NO: 1 - protein, 15 aa, synthetic construct
  REGION 1..15: note = Human IgG1
EPKSCDKTHTCPPCP

SEQ ID NO: 2 - protein, 12 aa, synthetic construct
  REGION 1..12: note = Human IgG2
ERKCCVECPPCP

SEQ ID NO: 3 - protein, 61 aa, synthetic construct
  REGION 1..61: note = Human IgG3
ELKTPLGDTHTCPRCPEPKSCDTPPPCPRCPEPKSCDTPPPCPRCPEPKSCDTPPPCPRCP

SEQ ID NO: 4 - protein, 12 aa, synthetic construct
  REGION 1..12: note = Human IgG4
ESKYGPPCPSCP

SEQ ID NO: 5 - protein, 12 aa, synthetic construct
  REGION 1..12: note = Modified Human IgG4
ESKYGPPCPPCP

SEQ ID NO: 6 - protein, 12 aa, synthetic construct
  REGION 1..12: note = Modified Human IgG4
ESKYGPPCPPCP

SEQ ID NO: 7 - protein, 10 aa, synthetic construct
  REGION 1..10: note = Modified Human IgG4
KYGPPCPPCP

SEQ ID NO: 8 - protein, 13 aa, synthetic construct
  REGION 1..13: note = Modified Human IgG4
EVVKYGPPCPPCP

SEQ ID NO: 9 - protein, 12 aa, synthetic construct
  REGION 1..12: note = S spacer
ESKYGPPCPPCP

SEQ ID NO: 10 - protein, 119 aa, synthetic construct
  REGION 1..119: note = M spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
  REGION 1..229: note = L spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 84 bp, synthetic construct
  misc_feature 1..84: note = CD28tm
  source 1..84: mol_type = other DNA
atgttctgggtgctggtggtggtcggaggcgtgctggcctgctacagcctgctggtcaccgtggccttcatcatcttttgggtg

SEQ ID NO: 13 - protein, 28 aa, synthetic construct
  REGION 1..28: note = CD28tm
MFWVLVVVGGVLACYSLLVTVAFIIFWV

SEQ ID NO: 14 - protein, 122 aa, synthetic construct
  REGION 1..122: note = Ab01-VH
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSS

SEQ ID NO: 15 - protein, 116 aa, synthetic construct
  REGION 1..116: note = Ab02-VH
EVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSS

SEQ ID NO: 16 - protein, 107 aa, synthetic construct
  REGION 1..107: note = Ab01-VL
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIK

SEQ ID NO: 17 - protein, 108 aa, synthetic construct
  REGION 1..108: note = Ab02-VL
DIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIK

SEQ ID NO: 18 - protein, 122 aa, synthetic construct; identical to SEQ ID NO: 14
  REGION 1..122: note = VH region

SEQ ID NO: 19 - protein, 107 aa, synthetic construct; identical to SEQ ID NO: 16
  REGION 1..107: note = VL region

SEQ ID NO: 20 - protein, 6 aa, synthetic construct
  REGION 1..6: note = heavy chain CDR1
SRNGMS

SEQ ID NO: 21 - protein, 17 aa, synthetic construct
  REGION 1..17: note = heavy chain CDR2
TVSSGGSYIYYADSVKG

SEQ ID NO: 22 - protein, 13 aa, synthetic construct
  REGION 1..13: note = heavy chain CDR3
QGTTALATRFFDV

SEQ ID NO: 23 - protein, 11 aa, synthetic construct
  REGION 1..11: note = light chain CDR1
KASQDVGTAVA

SEQ ID NO: 24 - protein, 7 aa, synthetic construct
  REGION 1..7: note = light chain CDR2
SASYRST

SEQ ID NO: 25 - protein, 9 aa, synthetic construct
  REGION 1..9: note = light chain CDR3
QHHYSAPWT

SEQ ID NO: 26-27 - skipped (2 entries)

SEQ ID NO: 28 - protein, 17 aa, synthetic construct
  REGION 1..17: note = heavy chain CDR2
TVSSGGSYIYYADSVKG

SEQ ID NO: 29 - protein, 13 aa, synthetic construct
  REGION 1..13: note = heavy chain CDR3
QGTTALATRFFDV

SEQ ID NO: 30 - protein, 11 aa, synthetic construct
  REGION 1..11: note = light chain CDR1
KASQDVGTAVA

SEQ ID NO: 31 - protein, 7 aa, synthetic construct
  REGION 1..7: note = light chain CDR2
SASYRST

SEQ ID NO: 32 - protein, 9 aa, synthetic construct
  REGION 1..9: note = light chain CDR3
QHHYSAPWT

SEQ ID NO: 33 - protein, 7 aa, synthetic construct
  REGION 1..7: note = heavy chain CDR1
GFTFSRN

SEQ ID NO: 34 - protein, 10 aa, synthetic construct
  REGION 1..10: note = heavy chain CDR1
GFTFSRNGMS

SEQ ID NO: 35 - protein, 5 aa, synthetic construct
  REGION 1..5: note = heavy chain CDR1
RNGMS

SEQ ID NO: 36 - protein, 6 aa, synthetic construct
  REGION 1..6: note = heavy chain CDR1
SRNGMS

SEQ ID NO: 37 - protein, 6 aa, synthetic construct
  REGION 1..6: note = heavy chain CDR2
SSGGSY

SEQ ID NO: 38 - protein, 10 aa, synthetic construct
  REGION 1..10: note = heavy chain CDR2
TVSSGGSYIY

SEQ ID NO: 39 - protein, 10 aa, synthetic construct
  REGION 1..10: note = heavy chain CDR2
TVSSGGSYIY

SEQ ID NO: 40 - protein, 17 aa, synthetic construct
  REGION 1..17: note = heavy chain CDR2
TVSSGGSYIYYADSVKG

SEQ ID NO: 41 - protein, 15 aa, synthetic construct
  REGION 1..15: note = heavy chain CDR3
ARQGTTALATRFFDV

SEQ ID NO: 42 - protein, 452 aa, synthetic construct
  REGION 1..452: note = Fc region
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSCSPGK

SEQ ID NO: 43 - protein, 452 aa, synthetic construct
  REGION 1..452: note = Fc region
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNCFSCSVMHEALHNHYTQKSLSCSPGK

SEQ ID NO: 44 - protein, 452 aa, synthetic construct
  REGION 1..452: note = Fc region
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 45 - protein, 448 aa, synthetic construct
  REGION 1..448: note = Fc region
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPCVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 46 - protein, 214 aa, synthetic construct
  REGION 1..214: note = Fc region
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 47 - protein, 214 aa, synthetic construct
  REGION 1..214: note = Fc region
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKRTVACPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 106 aa, synthetic construct
  REGION 1..106: note = Fc region
TVAAPSVFIFPPSDEQLKSGTASVVCLLNNFYPREAKVQWKVDNALQSGNSQESVTEQDSKDSTYSLSSTLTLSKAAYEKHKVYACEVTHQGLSSPVTKSFNRGEC

SEQ ID NO: 49 - protein, 12 aa, synthetic construct
  REGION 1..12: note = LCDR1
TASLSVSSTYLH

SEQ ID NO: 50 - protein, 7 aa, synthetic construct
  REGION 1..7: note = LCDR2
STSNLAS

SEQ ID NO: 51 - protein, 9 aa, synthetic construct
  REGION 1..9: note = LCDR3
HQYHRSPLT

SEQ ID NO: 52 - protein, 11 aa, synthetic construct
  REGION 1..11: note = LCDR1
KASQDVGTAVA

SEQ ID NO: 53 - protein, 7 aa, synthetic construct
  REGION 1..7: note = LCDR2
SASYRST

SEQ ID NO: 54 - protein, 9 aa, synthetic construct
  REGION 1..9: note = LCDR3
QHHYSAPWT

SEQ ID NO: 55 - protein, 6 aa, synthetic construct
  REGION 1..6: note = HCDR1
TKYGVH

SEQ ID NO: 56 - protein, 16 aa, synthetic construct
  REGION 1..16: note = HCDR2
VKWAGGSTDYNSALMS

SEQ ID NO: 57 - protein, 8 aa, synthetic construct
  REGION 1..8: note = HCDR3
DHRDAMDY

SEQ ID NO: 58 - protein, 6 aa, synthetic construct
  REGION 1..6: note = HCDR1
SRNGMS

SEQ ID NO: 59 - protein, 17 aa, synthetic construct
  REGION 1..17: note = HCDR2
TVSSGGSYIYYADSVKG

SEQ ID NO: 60 - protein, 13 aa, synthetic construct
  REGION 1..13: note = HCDR3
QGTTALATRFFDV

SEQ ID NO: 61 - DNA, 830 bp, synthetic construct
  misc_feature 1..830: note = IL13Ra2 Ab01 VLVH scFv
  source 1..830: mol_type = other DNA
gctagcccgccaccatgcttctcctggtgacaagccttctgctctgtgagttaccacacccagcattcctcctgatcccagacatccagatgacccagtccccctcttctctgtctgcctctgtgggcgacagagtgaccatcacctgtaaggccagtcaggatgtaggtactgctgtagcctggtatcagcagaagcctggcaaggctcccaagctgctgatctactcggcatcctaccggtccactggcgtgccttccagattctccggctctggctctggcaccgatttcaccctgaccatctcctccctccagcctgaggatttcgccacctactactgccagcaccattatagtgctccgtggacgtttggcggcggaacaaaggtggagatcaagggtggtggtggttctggcggcggcggctccggtggtggtggttctgaggtgcagctggtggagtctggcggcggactggtgcagcctggcggctctctgagactgtcttgtgccgcctccggcttcaccttcagtaggaatggcatgtcttgggtgaggcaggcccctggcaagggcctggagtgggtggccaccgttagtagtggtggtagttacatctactatgcagacagtgtgaaggggcggttcaccatctccagggacaacgccaagaactccctgtacctccagatgaactccctgagggccgaggataccgccgtgtactactgtgccagacaagggactacggcactagctacgaggttcttcgatgtctggggccagggcaccctggtgaccgtgtcctctgaatctaagtacggaccg

SEQ ID NO: 62 - DNA, 830 bp, synthetic construct
  misc_feature 1..830: note = IL13Ra2 Ab01 VHVL scFv Ab01-VL sequence Ab01-VH sequence ATG Start codon Gctagc = 5'-NheI restriction site; Cggaccg = 3'-RsrII restriction site
  source 1..830: mol_type = other DNA
gctagcccgccaccatgcttctcctggtgacaagccttctgctctgtgagttaccacacccagcattcctcctgatcccagaggtgcagctggtggagtctggcggcggactggtgcagcctggcggctctctgagactgtcttgtgccgcctccggcttcaccttcagtaggaatggcatgtcttgggtgaggcaggcccctggcaagggcctggagtgggtggccaccgttagtagtggtggtagttacatctactatgcagacagtgtgaaggggcggttcaccatctccagggacaacgccaagaactccctgtacctccagatgaactccctgagggccgaggataccgccgtgtactactgtgccagacaagggactacggcactagctacgaggttcttcgatgtctggggccagggcaccctggtgaccgtgtcctctggtggtggtggttctggcggcggcggctccggtggtggtggttctgacatccagatgacccagtccccctcttctctgtctgcctctgtgggcgacagagtgaccatcacctgtaaggccagtcaggatgtaggtactgctgtagcctggtatcagcagaagcctggcaaggctcccaagctgctgatctactcggcatcctaccggtccactggcgtgccttccagattctccggctctggctctggcaccgatttcaccctgaccatctcctccctccagcctgaggatttcgccacctactactgccagcaccattatagtgctccgtggacgtttggcggcggaacaaaggtggagatcaaggaatctaagtacggaccg

SEQ ID NO: 63 - DNA, 815 bp, synthetic construct
  misc_feature 1..815: note = IL13Ra2 Ab02 VLVH scFv
  source 1..815: mol_type = other DNA
gctagcccgccaccatgcttctcctggtgacaagccttctgctctgtgagttaccacacccagcattcctcctgatcccagatattcagatgacccagagcccgagcagcctgagcgcgagcgtgggcgatcgcgtgaccattacctgcaccgcgagcctgagcgtgagcagcacctatctgcattggtatcagcagaaaccgggcaaagcgccgaaactgctgatttatagcaccagcaacctggcgagcggcgtgccgagccgctttagcggcagcggcagcggcaccgattttaccctgaccattagcagcctgcagccggaagattttgcgacctattattgccatcagtatcatcgcagcccgctgacctttggcggcggcaccaaagtggaaattaaaggtggtggtggttctggcggcggcggctccggtggtggtggttctgaagtgcagctggtggaaagcggcggcggcctggtgcagccgggcggcagcctgcgcctgagctgcgcggcgagcggctttacctttaccaaatatggcgtgcattgggtgcgccaggcgccgggcaaaggcctggaatgggtggcggtgaaatgggcgggcggcagcaccgattataacagcgcgctgatgagccgctttaccattagccgcgataacgcgaaaaacagcctgtatctgcagatgaacagcctgcgcgcggaagataccgcggtgtattattgcgcgcgcgatcatcgcgatgcgatggattattggggccagggcaccctggtgaccgtgagcagcgaatctaagtacggaccg

SEQ ID NO: 64 - DNA, 815 bp, synthetic construct
  misc_feature 1..815: note = IL13Ra2 Ab02 VHVL scFv Ab02-VL sequence Ab02-VH sequence ATG Start codon Gctagc = 5'-NheI restriction site; Cggaccg = 3'-RsrII restriction site
  source 1..815: mol_type = other DNA
gctagcccgccaccatgcttctcctggtgacaagccttctgctctgtgagttaccacacccagcattcctcctgatcccagaagtgcagctggtggaaagcggcggcggcctggtgcagccgggcggcagcctgcgcctgagctgcgcggcgagcggctttacctttaccaaatatggcgtgcattgggtgcgccaggcgccgggcaaaggcctggaatgggtggcggtgaaatgggcgggcggcagcaccgattataacagcgcgctgatgagccgctttaccattagccgcgataacgcgaaaaacagcctgtatctgcagatgaacagcctgcgcgcggaagataccgcggtgtattattgcgcgcgcgatcatcgcgatgcgatggattattggggccagggcaccctggtgaccgtgagcagcggtggtggtggttctggcggcggcggctccggtggtggtggttctgatattcagatgacccagagcccgagcagcctgagcgcgagcgtgggcgatcgcgtgaccattacctgcaccgcgagcctgagcgtgagcagcacctatctgcattggtatcagcagaaaccgggcaaagcgccgaaactgctgatttatagcaccagcaacctggcgagcggcgtgccgagccgctttagcggcagcggcagcggcaccgattttaccctgaccattagcagcctgcagccggaagattttgcgacctattattgccatcagtatcatcgcagcccgctgacctttggcggcggcaccaaagtggaaattaaagaatctaagtacggaccg

SEQ ID NO: 65 - protein, 122 aa, synthetic construct; identical to SEQ ID NO: 14
  REGION 1..122: note = Ab01-VH

SEQ ID NO: 66 - protein, 6 aa, synthetic construct
  REGION 1..6: note = Ab01 HCDR1
SRNGMS

SEQ ID NO: 67 - protein, 17 aa, synthetic construct
  REGION 1..17: note = Ab01 HCDR2
TVSSGGSYIYYADSVKG

SEQ ID NO: 68 - protein, 13 aa, synthetic construct
  REGION 1..13: note = Ab01 HCDR3
QGTTALATRFFDV

SEQ ID NO: 69 - protein, 107 aa, synthetic construct; identical to SEQ ID NO: 16
  REGION 1..107: note = Ab01-VL

SEQ ID NO: 70 - protein, 11 aa, synthetic construct
  REGION 1..11: note = Ab01 LCDR1
KASQDVGTAVA

SEQ ID NO: 71 - protein, 7 aa, synthetic construct
  REGION 1..7: note = Ab01 LCDR2
SASYRST

SEQ ID NO: 72 - protein, 9 aa, synthetic construct
  REGION 1..9: note = Ab01 LCDR3
QHHYSAPWT

SEQ ID NO: 73 - protein, 116 aa, synthetic construct; identical to SEQ ID NO: 15
  REGION 1..116: note = Ab02-VH

SEQ ID NO: 74 - protein, 6 aa, synthetic construct
  REGION 1..6: note = Ab02 HCDR1
TKYGVH

SEQ ID NO: 75 - protein, 16 aa, synthetic construct
  REGION 1..16: note = Ab02 HCDR2
VKWAGGSTDYNSALMS

SEQ ID NO: 76 - protein, 8 aa, synthetic construct
  REGION 1..8: note = Ab02 HCDR3
DHRDAMDY

SEQ ID NO: 77 - protein, 108 aa, synthetic construct; identical to SEQ ID NO: 17
  REGION 1..108: note = Ab02-VL

SEQ ID NO: 78 - protein, 12 aa, synthetic construct
  REGION 1..12: note = Ab02 LCDR1
TASLSVSSTYLH

SEQ ID NO: 79 - protein, 7 aa, synthetic construct
  REGION 1..7: note = Ab02 LCDR2
STSNLAS

SEQ ID NO: 80 - protein, 9 aa, synthetic construct
  REGION 1..9: note = Ab02 LCDR3
HQYHRSPLT

SEQ ID NO: 81 - protein, 187 aa, synthetic construct
  REGION 1..187: note = DHFRdm
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 82 - DNA, 564 bp, synthetic construct
  misc_feature 1..564: note = DHFRdm
  source 1..564: mol_type = other DNA
atggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaa

SEQ ID NO: 83 - DNA, 66 bp, synthetic construct
  misc_feature 1..66: note = GMCSFss Leader
  source 1..66: mol_type = other DNA
atgcttctcctggtgacaagccttctgctctgtgagttaccacacccagcattcctcctgatccca

SEQ ID NO: 84 - DNA, 321 bp, synthetic construct
  misc_feature 1..321: note = Ab01-VL
  source 1..321: mol_type = other DNA
gacatccagatgacccagtccccctcttctctgtctgcctctgtgggcgacagagtgaccatcacctgtaaggccagtcaggatgtaggtactgctgtagcctggtatcagcagaagcctggcaaggctcccaagctgctgatctactcggcatcctaccggtccactggcgtgccttccagattctccggctctggctctggcaccgatttcaccctgaccatctcctccctccagcctgaggatttcgccacctactactgccagcaccattatagtgctccgtggacgtttggcggcggaacaaaggtggagatcaag

SEQ ID NO: 85 - DNA, 366 bp, synthetic construct
  misc_feature 1..366: note = Ab01-VH
  source 1..366: mol_type = other DNA
gaggtgcagctggtggagtctggcggcggactggtgcagcctggcggctctctgagactgtcttgtgccgcctccggcttcaccttcagtaggaatggcatgtcttgggtgaggcaggcccctggcaagggcctggagtgggtggccaccgttagtagtggtggtagttacatctactatgcagacagtgtgaaggggcggttcaccatctccagggacaacgccaagaactccctgtacctccagatgaactccctgagggccgaggataccgccgtgtactactgtgccagacaagggactacggcactagctacgaggttcttcgatgtctggggccagggcaccctggtgaccgtgtcctct

SEQ ID NO: 86 - DNA, 324 bp, synthetic construct
  misc_feature 1..324: note = Ab02-VL
  source 1..324: mol_type = other DNA
gatattcagatgacccagagcccgagcagcctgagcgcgagcgtgggcgatcgcgtgaccattacctgcaccgcgagcctgagcgtgagcagcacctatctgcattggtatcagcagaaaccgggcaaagcgccgaaactgctgatttatagcaccagcaacctggcgagcggcgtgccgagccgctttagcggcagcggcagcggcaccgattttaccctgaccattagcagcctgcagccggaagattttgcgacctattattgccatcagtatcatcgcagcccgctgacctttggcggcggcaccaaagtggaaattaaa

SEQ ID NO: 87 - DNA, 348 bp, synthetic construct
  misc_feature 1..348: note = Ab02-VH
  source 1..348: mol_type = other DNA
gaagtgcagctggtggaaagcggcggcggcctggtgcagccgggcggcagcctgcgcctgagctgcgcggcgagcggctttacctttaccaaatatggcgtgcattgggtgcgccaggcgccgggcaaaggcctggaatgggtggcggtgaaatgggcgggcggcagcaccgattataacagcgcgctgatgagccgctttaccattagccgcgataacgcgaaaaacagcctgtatctgcagatgaacagcctgcgcgcggaagataccgcggtgtattattgcgcgcgcgatcatcgcgatgcgatggattattggggccagggcaccctggtgaccgtgagcagc

SEQ ID NO: 88 - DNA, 45 bp, synthetic construct
  misc_feature 1..45: note = (Gly-Gly-Gly-Ser)x3 linker
  source 1..45: mol_type = other DNA
ggtggtggtggttctggcggcggcggctccggtggtggtggttct

SEQ ID NO: 89 - DNA, 732 bp, synthetic construct
  misc_feature 1..732: note = Ab01 scFv VLVH
  source 1..732: mol_type = other DNA
gacatccagatgacccagtccccctcttctctgtctgcctctgtgggcgacagagtgaccatcacctgtaaggccagtcaggatgtaggtactgctgtagcctggtatcagcagaagcctggcaaggctcccaagctgctgatctactcggcatcctaccggtccactggcgtgccttccagattctccggctctggctctggcaccgatttcaccctgaccatctcctccctccagcctgaggatttcgccacctactactgccagcaccattatagtgctccgtggacgtttggcggcggaacaaaggtggagatcaagggtggtggtggttctggcggcggcggctccggtggtggtggttctgaggtgcagctggtggagtctggcggcggactggtgcagcctggcggctctctgagactgtcttgtgccgcctccggcttcaccttcagtaggaatggcatgtcttgggtgaggcaggcccctggcaagggcctggagtgggtggccaccgttagtagtggtggtagttacatctactatgcagacagtgtgaaggggcggttcaccatctccagggacaacgccaagaactccctgtacctccagatgaactccctgagggccgaggataccgccgtgtactactgtgccagacaagggactacggcactagctacgaggttcttcgatgtctggggccagggcaccctggtgaccgtgtcctct

SEQ ID NO: 90 - DNA, 732 bp, synthetic construct
  misc_feature 1..732: note = Ab01 scFv VHVL
  source 1..732: mol_type = other DNA
gaggtgcagctggtggagtctggcggcggactggtgcagcctggcggctctctgagactgtcttgtgccgcctccggcttcaccttcagtaggaatggcatgtcttgggtgaggcaggcccctggcaagggcctggagtgggtggccaccgttagtagtggtggtagttacatctactatgcagacagtgtgaaggggcggttcaccatctccagggacaacgccaagaactccctgtacctccagatgaactccctgagggccgaggataccgccgtgtactactgtgccagacaagggactacggcactagctacgaggttcttcgatgtctggggccagggcaccctggtgaccgtgtcctctggtggtggtggttctggcggcggcggctccggtggtggtggttctgacatccagatgacccagtccccctcttctctgtctgcctctgtgggcgacagagtgaccatcacctgtaaggccagtcaggatgtaggtactgctgtagcctggtatcagcagaagcctggcaaggctcccaagctgctgatctactcggcatcctaccggtccactggcgtgccttccagattctccggctctggctctggcaccgatttcaccctgaccatctcctccctccagcctgaggatttcgccacctactactgccagcaccattatagtgctccgtggacgtttggcggcggaacaaaggtggagatcaag

SEQ ID NO: 91 - DNA, 717 bp, synthetic construct
  misc_feature 1..717: note = Ab02 scFv VLVH
  source 1..717: mol_type = other DNA
gatattcagatgacccagagcccgagcagcctgagcgcgagcgtgggcgatcgcgtgaccattacctgcaccgcgagcctgagcgtgagcagcacctatctgcattggtatcagcagaaaccgggcaaagcgccgaaactgctgatttatagcaccagcaacctggcgagcggcgtgccgagccgctttagcggcagcggcagcggcaccgattttaccctgaccattagcagcctgcagccggaagattttgcgacctattattgccatcagtatcatcgcagcccgctgacctttggcggcggcaccaaagtggaaattaaaggtggtggtggttctggcggcggcggctccggtggtggtggttctgaagtgcagctggtggaaagcggcggcggcctggtgcagccgggcggcagcctgcgcctgagctgcgcggcgagcggctttacctttaccaaatatggcgtgcattgggtgcgccaggcgccgggcaaaggcctggaatgggtggcggtgaaatgggcgggcggcagcaccgattataacagcgcgctgatgagccgctttaccattagccgcgataacgcgaaaaacagcctgtatctgcagatgaacagcctgcgcgcggaagataccgcggtgtattattgcgcgcgcgatcatcgcgatgcgatggattattggggccagggcaccctggtgaccgtgagcagc

SEQ ID NO: 92 - DNA, 717 bp, synthetic construct
  misc_feature 1..717: note = Ab02 scFv VHVL
  source 1..717: mol_type = other DNA
gaagtgcagctggtggaaagcggcggcggcctggtgcagccgggcggcagcctgcgcctgagctgcgcggcgagcggctttacctttaccaaatatggcgtgcattgggtgcgccaggcgccgggcaaaggcctggaatgggtggcggtgaaatgggcgggcggcagcaccgattataacagcgcgctgatgagccgctttaccattagccgcgataacgcgaaaaacagcctgtatctgcagatgaacagcctgcgcgcggaagataccgcggtgtattattgcgcgcgcgatcatcgcgatgcgatggattattggggccagggcaccctggtgaccgtgagcagcggtggtggtggttctggcggcggcggctccggtggtggtggttctgatattcagatgacccagagcccgagcagcctgagcgcgagcgtgggcgatcgcgtgaccattacctgcaccgcgagcctgagcgtgagcagcacctatctgcattggtatcagcagaaaccgggcaaagcgccgaaactgctgatttatagcaccagcaacctggcgagcggcgtgccgagccgctttagcggcagcggcagcggcaccgattttaccctgaccattagcagcctgcagccggaagattttgcgacctattattgccatcagtatcatcgcagcccgctgacctttggcggcggcaccaaagtggaaattaaa

SEQ ID NO: 93 - DNA, 36 bp, synthetic construct
  misc_feature 1..36: note = S spacer
  source 1..36: mol_type = other DNA
gaatctaagtacggaccgccctgccccccttgccct

SEQ ID NO: 94 - DNA, 84 bp, synthetic construct; identical to SEQ ID NO: 12
  misc_feature 1..84: note = CD28tm
  source 1..84: mol_type = other DNA

SEQ ID NO: 95 - DNA, 126 bp, synthetic construct
  misc_feature 1..126: note = 41-BB
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 96 - DNA, 336 bp, synthetic construct
  misc_feature 1..336: note = CD3?
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 97 - DNA, 72 bp, synthetic construct
  misc_feature 1..72: note = T2A
  source 1..72: mol_type = other DNA
ctcgagggcggcggagagggcagaggaagtcttctaacatgcggtgacgtggaggagaatcccggccctagg

SEQ ID NO: 98 - DNA, 1005 bp, synthetic construct
  misc_feature 1..1005: note = EGFRt
  source 1..1005: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 99 - DNA, 564 bp, synthetic construct; identical to SEQ ID NO: 82
  misc_feature 1..564: note = DHFRdm
  source 1..564: mol_type = other DNA

SEQ ID NO: 100 - DNA, 357 bp, synthetic construct
  misc_feature 1..357: note = M spacer
  source 1..357: mol_type = other DNA
gaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 101 - DNA, 685 bp, synthetic construct
  misc_feature 1..685: note = L spacer
  source 1..685: mol_type = other DNA
atctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

(19) United States
(12) Patent Application Publication
(10) Pub. No.: US 2025/0043013 A1
(43) Pub. Date: Feb. 6, 2025

(54) NUCLEIC ACIDS ENCODING IL-13 RECEPTOR ALPHA 2 (IL13RA2) CHIMERIC ANTIGEN RECEPTOR FOR TUMOR SPECIFIC T CELL IMMUNOTHERAPY

(71) Applicant: Seattle Children’s Hospital d/b/a Seattle Children’s Research Institute, Seattle, WA (US)

(72) Inventors: Giacomo Tampella, Seattle, WA (US); Michael C. Jensen, Bainbridge Island, WA (US)

(73) Assignee: Seattle Children’s Hospital d/b/a Seattle Children’s Research Institute, Seattle, WA (US)

(21) Appl. No.: 18/917,176

(22) Filed: Oct. 16, 2024

Related U.S. Application Data
(62) Division of application No. 16/979,471, filed on Sep. 9, 2020, now Pat. No. 12,152,079.
(60) Provisional application No. 62/643,055, filed on Mar. 14, 2018.

(51) Int. Cl. C07K 16/28; A61K 39/00; A61K 39/395; A61P 35/00; C07K 14/705; C07K 14/725; C12N 5/0783

(52) U.S. Cl. CPC C07K 16/2866; A61K 39/3955; A61K 39/4611; A61K 39/4631; A61K 39/464419; A61K 2239/31; A61K 2239/38; A61K 2239/47; A61P 35/00; C07K 14/7051; C07K 14/70521; C07K 14/70578; C12N 5/0636; C07K 2319/03

(57) ABSTRACT

Some embodiments of the methods and compositions provided herein relate to chimeric antigen receptors (CARs) that specifically bind to human extracellular domains of the IL-13 alpha 2 (IL13Ra2) receptor, cells containing such CARs, and methods of cell-based immunotherapy targeting cancer cells, such as cells of solid tumors.

### CROSS-REFERENCE TO RELATED APPLICATIONS

This application is a divisional of U.S. patent application Ser. No. 16/979,471, filed on Sep. 9, 2020, which is a U.S. national stage entry of PCT/US2019/021833, filed Mar. 12, 2019, which claims priority to U.S. Prov. App. No. 62/643,055 filed Mar. 14, 2018, which are hereby expressly incorporated by reference in their entirety.

### REFERENCE TO SEQUENCE LISTING

The Sequence Listing associated with this application is provided in XML format in lieu of a paper copy and is hereby incorporated by reference into the specification. The name of the file containing the Sequence Listing is S281-0020USC1_seq.xml. The file is 139,544 bytes, was created Oct. 15, 2024, and is being submitted electronically via Patent Center.

### FIELD OF THE INVENTION

Some embodiments of the methods and compositions provided herein relate to chimeric antigen receptors (CARs) that specifically bind to human extracellular domains of an IL-13 alpha 2 (IL13Ra2) receptor, to cells containing such CARs, and to methods of cell-based immunotherapy targeting cancer cells, such as cells of solid tumors.

### BACKGROUND OF THE INVENTION

Despite significant advances in the understanding of brain cancer, during the last decade, the mortality rate has remained consistent and new innovative therapies are urgently needed. To date, T cell immunotherapy has emerged as a promising cancer therapy supported by remarkable clinical data reporting complete remission in patients with B cell malignancies after administration of T cell CARs targeting CD19. However, there remains a need for further and improved T cell immunotherapies.

### SUMMARY OF THE INVENTION

Some embodiments of the methods and compositions provided herein include a nucleic acid encoding a chimeric antigen receptor, the chimeric antigen receptor comprising: a ligand binding domain that binds to and/or interacts with an IL-13 alpha 2 (IL13Ra2) receptor; a polypeptide spacer between the ligand binding domain and a transmembrane domain; the transmembrane domain; and intracellular signaling region.

In some embodiments, the ligand binding domain comprises: a heavy chain complementarity determining region 1 (CDR1) having the amino acid sequence of SEQ ID NO:20, or conservative variations thereof; a heavy chain complementarity determining region 2 (CDR2) having the amino acid sequence of SEQ ID NO:21, or conservative variations thereof; and/or a heavy chain complementarity determining region 3 (CDR3) having the amino acid sequence of SEQ ID NO:22, or conservative variations thereof.

In some embodiments, the ligand binding domain comprises: a light chain complementarity determining region 1 (CDR1) having the amino acid sequence of SEQ ID NO:23, or conservative variations thereof; a light chain complementarity determining region 2 (CDR2) having the amino acid sequence of SEQ ID NO:24, or conservative variations thereof; and/or a light chain complementarity determining region 3 (CDR3) having the amino acid sequence of SEQ ID NO:25, or conservative variations thereof.

In some embodiments, the ligand binding domain comprises a heavy chain variable (VH) domain comprising a polypeptide having at least 90% identity with the amino acid sequence of SEQ ID NO:18.

In some embodiments, the ligand binding domain comprises a light chain variable (VL) domain comprising a polypeptide having at least 90% identity with the amino acid sequence of SEQ ID NO:19.

In some embodiments, wherein the ligand binding domain comprises: a heavy chain CDR1 having the amino acid sequence of SEQ ID NO:20; a heavy chain CDR2 having the amino acid sequence of SEQ ID NO:21; and/or a heavy chain CDR3 having the amino acid sequence of SEQ ID NO:22.

In some embodiments, the ligand binding domain comprises: a light chain CDR1 having the amino acid sequence of SEQ ID NO:23; a light chain CDR2 having the amino acid sequence of SEQ ID NO:24; and/or a light chain CDR3 having the amino acid sequence of SEQ ID NO:25.

In some embodiments, the ligand binding domain comprises a VH domain comprising a polypeptide having the amino acid sequence of SEQ ID NO:18.

In some embodiments, the ligand binding domain comprises a VL domain having the amino acid sequence of SEQ ID NO:19.

In some embodiments, the ligand binding domain is an antibody fragment, such as an antigen-binding fragment.

In some embodiments, the ligand binding domain is a single chain variable fragment (scFv). In some embodiments, the scFv comprises a VL-VH orientation. In some embodiments, the single chain variable fragment (scFv) comprises a sequence having at least 95% identity with the nucleotide sequence of SEQ ID NO:61. In some embodiments, the single chain variable fragment (scFv) comprises the nucleotide sequence of SEQ ID NO:61.

In some embodiments, the spacer comprises an amino acid sequence of X₁PPX₂P.

In some embodiments, the spacer region comprises a portion of a hinge region of a human antibody or modified variant thereof.

In some embodiments, the spacer is 15 amino acids or less but not less than 1 or 2 amino acids.

In some embodiments, the spacer comprises, consists, or consists essentially of a sequence having at least 95% identity with the amino acid sequence of SEQ ID NO:09. In some embodiments, the spacer comprises, consists, or consists essentially of an amino acid sequence of SEQ ID NO:09, or conservative substitutions thereof.

In some embodiments, the spacer comprises, consists of, or consists essentially of, an IgG4 hinge spacer (S).

In some embodiments, the spacer comprises, consists of, or consists essentially of, an IgG4 hinge-CH3 spacer (M).

In some embodiments, the spacer comprises, consists of, or consists essentially of, an IgG4 hinge-CH2 (L234D, N297A)-CHE spacer (L).

In some embodiments, the intracellular signaling region comprises primary and a costimulatory signaling domains, optionally comprising all or a portion of a CD3 zeta in combination with a co-stimulatory domain selected from the group consisting of signaling domains of CD27, CD28, 4-1BB, OX-40, CD30, CD40, PD-1, ICOS, LFA-1, CD2, CD7, NKG2C, and B7-H3 and combinations thereof.

In some embodiments, the intracellular signaling region comprises a signaling portion of a CD3 zeta and a signaling portion of a 4-1BB.

In some embodiments, the transmembrane domain comprises a CD28 transmembrane domain (CD28tm).

Some embodiments also include a nucleic acid that encodes a marker sequence. In some embodiments, the marker sequence is a truncated receptor and optionally is an EGFRt, a HER2t, or a CD19t.

Some embodiments also include a dihydrofolate reductase transgene configured for methotrexate selection. In some embodiments, the dihydrofolate reductase transgene is a dihydrofolate reductase double mutant (DHFRdm). In some embodiments, the dihydrofolate reductase double mutant comprises amino acid mutations of L22F and F31S.

Some embodiments of the methods and compositions provided herein include an expression vector comprising the nucleic acid of any one of the embodiments provided herein. In some embodiments, the vector is a viral vector. In some embodiments, the vector is a lentiviral or adenoviral vector.

Some embodiments of the methods and compositions provided herein include a chimeric antigen receptor (CAR) polypeptide encoded by the nucleic acid of any one of the embodiments provided herein.

Some embodiments of the methods and compositions provided herein include a host cell comprising the nucleic acid of any one of the embodiments provided herein.

In some embodiments, the host cell is a CD8+ T cytotoxic lymphocyte cell selected from the group consisting of naïve CD8+ T cells, central memory CD8+ T cells, effector memory CD8+ T cells and bulk CD8+ T cells. In some embodiments, the CD8+ cytotoxic T lymphocyte cell is a central memory T cell and, wherein the central memory T cell is positive for CD45RO+, CD62L+, and CD8+.

In some embodiments, the host cell is a CD4+ T helper lymphocyte cell selected from the group consisting of naïve CD4+ T cells, central memory CD4+ T cells, effector memory CD4+ T cells, and bulk CD4+ T cells. In some embodiments, the CD4+ helper lymphocyte cell is a naïve CD4+ T cell and, wherein the naïve CD4+ T cell is positive for CD45RA+, CD62L+ and CD4+ and negative for CD45RO.

In some embodiments, the host cell is a precursor T cell.

In some embodiments, the host cell is a hematopoietic stem cell.

Some embodiments of the methods and compositions provided herein include a pharmaceutical composition comprising the host cell of any one of the embodiments provided herein, and a pharmaceutically acceptable excipient.

Some embodiments of the methods and compositions provided herein include a method for preparing the host cell of any one of the embodiments provided herein, comprising: introducing the nucleic acid of any one of the embodiments provided herein into a cell; culturing the cell in the presence of anti-CD3 antibody and/or anti CD28 antibody, and at least one homeostatic cytokine in conditions sufficient for the cells to expand; and selecting the cell with a selection reagent; wherein the selection reagent is configured to selectively enrich cells transduced with the nucleic acid or vector.

In some embodiments, the cell is a lymphocyte. In some embodiments, the lymphocyte has a CD45RA−, CD45RO+, and CD62L+ phenotype. In some embodiments, the lymphocyte is CD8+ or CD4+.

In some embodiments, the selection reagent is methotrexate.

In some embodiments, the cytokine is IL-15, Il-7 and/or Il-21.

Some embodiments also include introducing a second nucleic acid into the host cell, wherein the second nucleic acid encodes a marker protein. In some embodiments, the marker protein is EGFRt.

Some embodiments of the methods and compositions provided herein include a host cell of any one of the embodiments provided herein for use in a medicament or for use in the treatment or inhibition of a cancer or a solid tumor expressing an IL-13a2 receptor. In some embodiments, the cancer comprises a brain cancer. In some embodiments, the cancer is a glioma or glioblastoma tumor. In some embodiments, the cancer is glioblastoma multiforme (GBM). In some embodiments, the cancer is a glioma.

Some embodiments of the methods and compositions provided herein include a method of treating, inhibiting, or ameliorating a cancer in a subject, comprising: administering the host cell of any one of the embodiments provided herein to the subject in need thereof. In some embodiments, the cancer is a IL13Ra-positive malignancy. In some embodiments, the cancer is brain cancer. In some embodiments, the cancer is a glioma or glioblastoma tumor. In some embodiments, the cancer is a glioma. In some embodiments, the cancer is glioblastoma multiforme (GBM). In some embodiments, the subject is mammalian. In some embodiments, the subject is human.

Some embodiments also include administering an addiotional therapy selected from chemotherapy and radiation therapy. In some embodiments, the chemotherapeutic drug comprises electochemotherapy, alkylating agent, antimetabolite (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine), anti-tumor antibiotic, topoisomerase inhibitor, mitotic inhibitor, corticosteroid, DNA intercalating agent, or checkpoint inhibitor (checkpoint kinase CHK1, CHK2).

### BRIEF DESCRIPTION OF THE DRAWINGS

FIG. 1 depicts a schematic of certain embodiments of IL-13Ra2-targeting CARs, and components used for the construction of embodiments the IL-13Ra2-targeting CARs. IL-13Ra2 specific CARs included: one of a number of single-chain variable fragments (scFv) that specifically recognize an extracellular epitope of IL-13 alpha 2 receptor (IL-13Ra2) derived from one of two antibodies ‘hu08’ (Ab01) or ‘hu07’ (Ab02) and included a VH and VL domains with a linker therebetween; one of various spacer domains (marked “S”, “M” and “L”); a transmembrane domain derived from a human CD28 (CD28tm); a costimulatory domain derived from human 4-1BB; a CD3ζ-derived signaling domain; a T2A ribosomal skip sequence; and a truncated EGFR (EG FRt) transduction marker; and optionally a further T2A ribosomal skip sequence and a dihydrofolate reductase double mutant (DHFRdm) transgene configured for methotrexate selection.

FIG. 2A depicts a flow cytometry analysis of EGFRt expression in CD8+ Tcells (upper panel) or CD4+ T cells (lower panel) transduced with various CARs depicted in FIG. 1. From the top row to the bottom row of each panel: T cell mock; T cell with IL-13Ra2 Ab01 VL-VH Spacer S CAR; T cell with IL-13Ra2 Ab01 VH-VL Spacer S CAR; T cell with IL-13Ra2 Ab02 VL-VH Spacer S CAR; and T cell with IL-13Ra2 Ab02 VH-VL Spacer S CAR.

FIG. 2B depicts graphs of cytokine production (IL-2, IFN-γ, TNF-α) from CD8+ T cells transduced with various CARs and incubated with indicated target cells. The key corresponds to T cell mock; T cell with IL-13Ra2 Ab01 VL-VH Spacer S CAR; T cell with IL-13Ra2 Ab01 VH-VL Spacer S CAR; T cell with IL-13Ra2 Ab02 VL-VH Spacer S CAR; and T cell with IL-13Ra2 Ab02 VH-VL Spacer S CAR.

FIG. 2C depicts graphs of cytokine production (IL-2, IFN-γ, TNF-α) from CD4+ T cells transduced with various CARs and incubated with indicated target cells. The key corresponds to T cell mock; T cell with IL-13Ra2 Ab01 VL-VH Spacer S CAR; T cell with IL-13Ra2 Ab01 VH-VL Spacer S CAR; T cell with IL-13Ra2 Ab02 VL-VH Spacer S CAR; and T cell with IL-13Ra2 Ab02 VH-VL Spacer S CAR.

FIG. 3A depicts a multi-parameter flow cytometry analysis of T cells derived from healthy donor and transduced with IL-13Ra2 hu08 CARs with histograms quadrants were drawn based on control staining (upper and middle panles); and a flow cytometry analysis of EGFRt expression in CD8+ T cells transduced with a CAR (lower panel). Indicated CARs included: mock; IL-13Ra2 Ab01 VL-VH Spacer-S CAR; IL-13Ra2 Ab01 VL-VH Spacer-M CAR; IL-13Ra2 Ab01 VL-VH Spacer-L CAR; IL-13Ra2 Ab01 VH-VL Spacer-S CAR; IL-13Ra2 Ab01 VH-VL Spacer-M CAR; and IL-13Ra2 Ab01 VH-VL Spacer-L CAR.

FIG. 3B depicts an analysis for cytokine production and cytolytic activity of CD8+ T cells containing a CAR, following co-culture with certain indicated target cells. Indicated CARs included: mock; IL-13Ra2 Ab01 VL-VH Spacer-S CAR; IL-13Ra2 Ab01 VL-VH Spacer-M CAR; IL-13Ra2 Ab01 VL-VH Spacer-L CAR; IL-13Ra2 Ab01 VH-VL Spacer-S CAR; IL-13Ra2 Ab01 VH-VL Spacer-M CAR; and IL-13Ra2 Ab01 VH-VL Spacer-L CAR.

FIG. 4A depicts graphs of total flux (photons/sec) as a measure of tumor burden (y-axis) over time (days post-tumor inoculation, x-axis), for mice treated with: vehicle, mock cells, 2×10⁶ T cells containing an anti-IL-13Ra2 CAR; 1×10⁶ T cells containing an anti-IL-13Ra2 CAR; or 0.5×10⁶ T cells containing an anti-IL-13Ra2 CAR. The anti-IL-13Ra2 CAR was IL-13Ra2 Ab01 VL-VH Spacer-S CAR.

FIG. 4B depicts a Kaplan-Meier survival curve for mice treated with increasing doses of T cells expressing an anti-IL-13Rα2 CAR (IL-13Ra2 Ab01 VL-VH Spacer-S CAR).

FIG. 5A depicts graphs of total flux (photons/sec) as a measure of tumor burden (y-axis) over time (days post-tumor inoculation, x-axis), for mice treated with: mock cells; 2×10⁶ T cells containing an anti-IL-13Rα2 CAR containing a short spacer; 2×10⁶ T cells containing an anti-IL-13Rα2 CAR containing a medium spacer; 2×10⁶ T cells containing an anti-IL-13Rα2 CAR containing a long spacer.

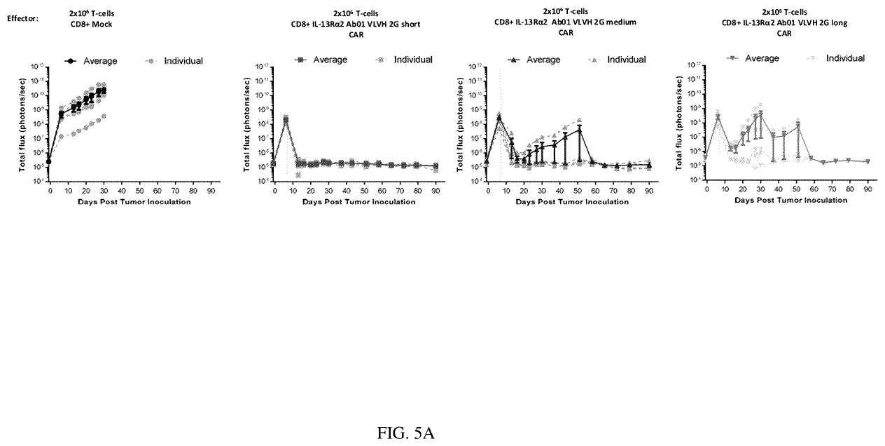

FIG. 5B depicts a Kaplan-Meier survival curve for mice treated with T cells expressing an anti-IL-13Rα2 CAR containing a long spacer, a medium spacer, or a short spacer.

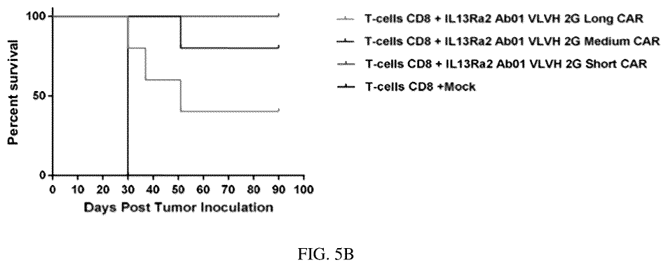

FIG. 5C depicts an area under curve (AUC) analysis of bioluminescent data for mice treated with T cells expressing an anti-IL-13Ra2 CAR containing a long spacer, a medium spacer, or a short spacer. The mean AUCs between IL13Rα2 Ab01 space variants CAR T-cells or Mock T-cells were compared. *, P<0.05. **, P<0.01. ****, P<0.0001.

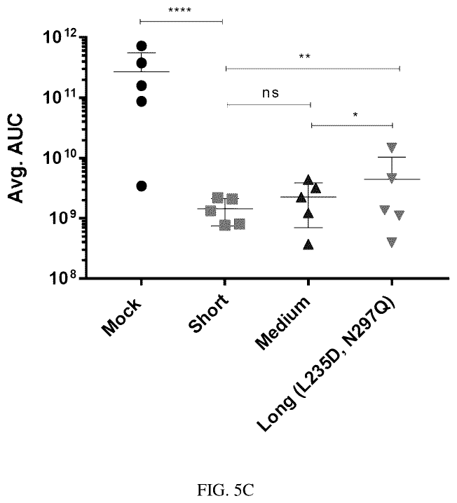

### DETAILED DESCRIPTION

Some embodiments of the methods and compositions provided herein include IL13Ra2-targeted chimeric antigen receptors (CARs), such as second-generation IL-13Rα2-specific CARs, cells containing such CARs, nucleic acids encoding such CARs, and related therapeutic methods and uses thereof. Among the provided CARs are those having particular combinations of components or domains, such as those resulting from optimization of CAR function. In some aspects, the IL13Ra2 CAR-targeted T cell therapeutics can also act as an alternative or supplement to current IL-13Rα2-specific and unspecific cancer treatments, such as combination therapies.

IL-13Ra2 was previously found to be abundant in metastatic or late-stage BLBC (Papageorgis et al. Breast Cancer Research, 2015; 17 (1); herein expressly incorporated by reference in its entirety). Based on publicly available data, correlations were made between, likelihood of progression-free survival based and high levels of IL-13Rα2. A subtype of BLBC that tended to spread to the lungs quickly was observed to have high IL-13Rα2 levels. IL-13Rα2 was also found to stimulate human glioma cell growth and metastasis through the Src/PI3K/Akt/mTOR signaling pathway. (Tu et al. Tumour Biol. 2016 Nov; 37(11):14701-14709; herein expressly incorporated by reference in its entirety). IL13Ra2 targeted therapies, such as chimeric receptor-based therapies, have been described (see, e.g., Brown et al Clin Cancer Res 2015; Brown et al N Engl J Med 2016; Brown et al Mol Ther 2017; WO 2014072888-A1, describing anti-IL-13 receptor alpha 2 (IL-13-Ra2) antibodies and antibody-drug conjugates for the treatment of cancer, each herein expressly incorporated by reference in their entirety). Among such therapies are those based on or including mutant IL13 (e.g., E13Y)-based binding or antigen recognition domains, such as zetakines.

In some embodiments, the provided CARs and associated methods and uses are based in part on observations described herein showing particularly advantageous activity and/or in vivo anti-cancer effects in the context of glioblastoma or model thereof, of CARs containing specific binding domains in combination with specific other components such as specific spacer domains curative. In some aspects, the provided compositions, methods and uses are employed in the treatment, inhibition, or prevention of glioblastoma and/or other IL-13Rα2-positive malignancies. In some aspects, the provided CARs described herein differ from other CARs in various respects, such as in one or more characteristics of their molecular structure, such as the IL-13Rα2 epitope specifically recognized by the binding domain and targeted by CARs provided herein.

The provided embodiments are based in part on observations herein, such as those demonstrated in preclinical studies described herein, demonstrating antitumor in vitro activity of provided CAR-T cells, against glioblastoma cell lines and the ability to reduce tumor growth in xenograft models, in some respects to a greater extent as compared with different CARs having different molecular structures. In light of these findings, the commercial use of compositions comprising these CARS in immunotherapy for brain tumors and other IL-13Rα2-positive types of cancer is contemplated.

### Definitions

As used herein, “nucleic acid” or “nucleic acid molecule” have their plain and ordinary meaning when read in light of the specification, and may include but is not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives, a nucleic acid sequence encoding a protein is provided. In some alternatives, the nucleic acid is RNA or DNA.

As used herein, “chimeric antigen receptor” has its plain and ordinary meaning when read in light of the specification, and may include but is not limited to, for example, a synthetically designed receptor comprising a ligand binding domain of an antibody or other protein sequence that binds to a molecule associated with the disease or disorder and is linked via a spacer domain to one or more intracellular signaling domains of a cell, such as a T cell, or other receptors, such as a costimulatory domain. Chimeric receptor can also be referred to as artificial cell receptors or T cell receptors, chimeric cell receptors or T cell receptors, chimeric immunoreceptors, and/or chimeric antigen receptors (CARs). These receptors can be used to graft the specificity of a monoclonal antibody or binding fragment thereof onto a cell, preferably a T-cell with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. The term chimeric antigen receptors or “CARs” are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

In some embodiments, the transmembrane domain is a region of a membrane-spanning protein that is hydrophobic that can reside in the bilayer of a cell to anchor a protein that is embedded to the biological membrane. Without being limiting, the topology of the transmembrane domain can be a transmembrane alpha helix. In some alternatives of the chimeric antigen receptor, the chimeric antigen receptor comprises a sequence encoding a transmembrane domain. In some alternatives, the transmembrane domain comprises a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some alternatives, the CD28 transmembrane sequence or fragment thereof comprise 28 amino acids in length.

In some embodiments, the signaling domains, such as primary signaling domains or costimulatory domains, include an intracellular or cytoplasmic domain of a protein or a receptor protein that interacts with components within the interior of the cells and is capable of relaying or participating in the relaying of a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes a co-stimulatory domain. In some aspects, the costimulatory domain includes a signaling moiety that provides to T-cells a signal which, in addition to the primary signal provided by for instance the CD3 zeta chain of the TCR/CD3 complex, enhances response such as a T-cell effector response, such as, for example, an immune response, activation, proliferation, differentiation, cytokine secretion, cytolytic activity, perforin and/or granzyme activity and the like. In some embodiments, the intracellular signaling domain and/or the co-stimulatory domain can include all or a portion of CD27, CD28, 4-1BB, OX40, CD30, CD40, ICOS, lymphocyte function-associated antigen-1 (LFA-1), CD2, CD7, LIGHT, NKG2C, and/or B7-H3, and/or a ligand that specifically binds with CD83.

As used herein, an “antibody” has its plain and ordinary meaning when read in light of the specification, and may include but is not limited to, for example, a large Y-shape protein produced by plasma cells that is used by the immune system to identify and neutralize foreign objects such as bacteria and viruses. In some contexts, the term antibody refers to antigen binding fragements of an antibody. The antibody protein can comprise four polypeptide chains; two identical heavy chains and two identical light chains connected by disulfide bonds. Each chain is composed of structural domains called immunoglobulin domains. These domains can contain 70-110 amino acids and are classified into different categories according to their size and function. A chimeric antigen receptor can comprise a ligand binding domain, which includes an antibody fragment, preferably an antigen binding fragment. In some alternatives, a nucleic acid encoding a chimeric antigen receptor (CAR) is provided, the nucleic acid comprising: a) a first polynucleotide encoding a ligand binding domain, wherein the ligand binding domain binds to and/or interacts with an IL-13 alpha 2 (IL13Rα2) receptor, b) a second polynucleotide encoding a polypeptide spacer of a length sufficient to allow the ligand binding domain to interact with the IL-13 alpha 2 (IL13Rα2) receptor, c) a third polynucleotide encoding a transmembrane domain and d) a fourth polynucleotide encoding an intracellular signaling domain. In some alternatives, the ligand binding domain is an antibody fragment.

As used herein, a “single chain variable fragment” or scFv has its plain and ordinary meaning when read in light of the specification, and may include but is not limited to, for example, a fusion protein that comprises the variable regions of the heavy chain (VH) and the light chains (VL) of an immunoglobulin, which are connected to one another with a short linker peptide. Without being limiting, the linker can comprise glycine for flexibility and hydrophilic amino acids, for example serine or threonine for solubility. The linker can connect the N-terminus of the VH with the C-terminus of the VL or it can connect the C-terminus of the VH with the N-terminus of the VL. In some alternatives, the ligand binding domain present on a CAR is a single chain variable fragment (scFv). In some alternatives, the scFv domain present on a CAR is specific for a IL-13 alpha 2 (IL13Rα2) receptor present on a tumor cell.

In some embodiments, the compositions, cells and vectors include marker sequences or nucleic acids encoding the same, which may include, for example, a protein that serves as a label for a cell. In some alternatives of the cells described herein, the cells co-express a marker protein for a specific chimeric antigen protein that is expressed. In some alternatives of the cells provided herein, the chimeric antigen receptor is co-expressed with a specific marker protein. In some alternatives of the cells provided herein, the cells comprise a nucleic acid encoding a chimeric antigen receptor. Markers may include selectable marker sequence, such as a gene introduced into a vector or a cell that confers a trait for artificial selection. A selectable marker sequence or marker sequence can be a screenable marker to allow a researcher to distinguish between wanted and unwanted cells, or to enrich for a specific cell type. In some alternatives, a vector is provided wherein the vector encodes a chimeric antigen receptor comprising a marker sequence, wherein said marker sequence encodes a cell surface selectable marker. In the alternatives described herein, the fusion protein provided can comprise a marker sequence that can be selected in experiments, such as flow cytometry. In some alternatives, the marker is the protein Her2tG or EGFRt.

Methotrexate (MTX) may include but is not limited to, for example, an antimetabolite and antifolate drug. In some aspects, it acts by inhibiting the metabolism of folic acid. In some alternatives, a method of generating engineered multiplexed T cells for adoptive T cell immunotherapy is provided. In the broadest sense, the method can comprise providing the gene delivery polynucleotide of any of the alternatives described herein, selecting the cells comprising the gene delivery polynucleotide, wherein the selecting comprises adding a selection reagent. In some alternatives described herein, the selection reagent comprises an agent for selection. In some alternatives, the selection reagent is MTX.

As used herein, “dihydrofolate reductase”, or DHFR, as described herein, has its plain and ordinary meaning when read in light of the specification, and may include but is not limited to, for example, an enzyme that reduces dihydrofolic acid to tetrahydrofolic acid, using NADPH as electron donor, which can be converted to the kinds of tetrahydrofolate cofactors used in 1-carbon transfer chemistry. In some alternatives described herein, a gene delivery polynucleotide is provided. In some alternatives, the gene delivery polynucleotide comprises at least one selectable marker cassette encoding for a double mutant of dihydrofolate reductase (DHFRdm).

In some embodiments, the constructs and sequences provided are modified or optimized, such as by codon optimization, which may include but is not limited to, for example, the design process of altering codons to codons known to increase maximum protein expression efficiency in a desired cell, preferably in a human cell. In some alternatives, codon optimization is described, wherein codon optimization can be performed by using algorithms that are known to those skilled in the art to create synthetic genetic transcripts optimized for high protein yield. Programs containing alogorithms for codon optimization are known to those skilled in the art. Programs can include, for example, OptimumGene™, GeneGPS® algorithms. Additionally, synthetic codon optimized sequences can be obtained commercially for example from Integrated DNA Technologies and other commercially available DNA sequencing services. In some alternatives, the nucleic acids are described, wherein the genes of the nucleic acid for the complete gene transcript are codon optimized for expression in humans. In some alternatives, the genes are optimized to have selected codons specifically for maximal protein expression in human cells, which can increase the concentration of proteins or CARs of a T cell.

Codon optimization can be performed to reduce the occurrence of secondary structure in a polynucleotide, as well. In some alternatives, codon optimization can also be performed to reduce the total GC/AT ratio. Strict codon optimization can also lead to unwanted secondary structure or an undesirable GC content that leads to secondary structure. As such the secondary structures affect transcriptional efficiency. Programs such as GeneOptimizer can be used after codon usage optimization, for secondary structure avoidance and GC content optimization. These additional programs can be used for further optimization and troubleshooting after an initial codon optimization to limit secondary structures that may occur after the first round of optimization. Alternative programs for optimization are known to those skilled in the art. In some alternatives, the nucleic acid comprises sequences that are codon optimized for expression in humans and/or to remove secondary structure and/or to reduce the total GC/AT ratio. In some alternatives, the sequences are optimized for secondary structure avoidance. In some alternatives, the sequences are optimized to reduce the total GC/AT ratio.

As used herein, “vector,” “expression vector” or “construct” has its plain and ordinary meaning when read in light of the specification, and may include but is not limited to, for example, a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some alternatives, the vectors are plasmid, minicircles, or viral genomes. In some alternatives, the vector is a viral vector. In some alternatives, the viral vector is a lentivirus. In some alternatives, the vector is a lentiviral vector. In some alternatives, the vector is a foamy viral vector, adenoviral vectors, retroviral vectors or lentiviral vectors.

T cells or T lymphocytes in some embodiments may include T cells from any mammalian, preferably primate, species, including monkeys, dogs, and humans. In some alternatives the T cells are allogeneic (from the same species but different donor) as the recipient subject who receives or is to receive the cells, such as in the form of a therapeutic composition; in some alternatives the T cells are autologous (the donor and the recipient are the same); in some alternatives the T cells arc syngeneic (the donor and the recipients are different but are identical twins).

As used herein, a “ribosome skip sequence” as described herein refers to a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some alternatives, the ribosome skip sequence is a P2A, T2A, E2A or F2A sequence. In some alternatives, the ribosome skip sequence is a T2A sequence.

Mature T cells express the surface protein CD4 and are referred to as CD4+ T cells. CD4+ T cells are generally treated as having a pre-defined role as helper T cells within the immune system. For example, when an antigen-presenting cell expresses an antigen on MHC class II, a CD4+ cell will aid those cells through a combination of cell to cell interactions (e.g. CD40 and CD40L) and through cytokines. Nevertheless, there are rare exceptions; for example, sub-groups of regulatory T cells, natural killer cells, and cytotoxic T cells express CD4. All of the latter CD4+ expressing T cell groups are not considered T helper cells.

As used herein, “central memory” T cell (or “T_{CM}”) has its plain and ordinary meaning when read in light of the specification, and may include but is not limited to, for example, an antigen experienced CTL that expresses CD62L or CCR-7 and CD45RO on the surface thereof, and does not express or has decreased expression of CD45RA as compared to naïve cells. In some alternatives, central memory cells are positive for expression of CD62L, CCR7, CD28, CD127, CD45RO, and/or CD95, and have decreased expression of CD54RA as compared to naïve cells.

As used herein, “effector” “T_{E}” T cells has its plain and ordinary meaning when read in light of the specification, and may include but is not limited to, for example, antigen experienced cytotoxic T lymphocyte cells that do not express or have decreased expression of CD62L, CCR7, or CD28, orare positive for granzyme B and/or perforin, as compared to central memory or naïve T cells.

As used herein, “pharmaceutical excipient,” or pharmaceutical vehicle has its plain and ordinary meaning when read in light of the specification, and may include but is not limited to, for example, a carrier or inert medium used as a solvent in which the medicinally active agent or T cells for treatment or therapy is formulated and or administered. Vehicles can include polymeric micelles, liposomes, lipoprotein-based carriers, nano-particle carriers, dendrimers, and/or other vehicles for T cells that are known to one skilled in the art. An ideal vehicle or excipient can be non-toxic, biocompatible, non-immunogenic, biodegradable, and can avoid recognition by the host's defense mechanisms.

As used herein, “T cell precursors” has its plain and ordinary meaning when read in light of the specification, and may include but is not limited to, for example, lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4⁻CD8⁻) cells. As they progress through their development, they become double-positive thymocytes (CD4⁺CD8⁺), and finally mature to single-positive (CD4⁺CD8⁻ or CD4⁻CD8⁺) thymocytes that are then released from the thymus to peripheral tissues.

Glioblastoma multiforme (GBM), generally is considered the most aggressive cancer that begins within the brain. Initial signs and symptoms of glioblastoma generally are non-specific. They may include headaches, personality changes, nausea, and symptoms similar to those of a stroke.

As used herein, a “leader sequence” also known as the “5’ untranslated region (5′ UTR), is the region of mRNA that is located upstream from the initiation codon and is important in regulating the translation of an mRNA transcript. In some alternatives of the method of making genetically modified T cells, which have a chimeric antigen receptor (CAR), the vector encoding the chimeric antigen receptor comprises a sequence encoding a leader sequence.

As described in the alternatives herein are ligands. “Ligand” has its plain and ordinary meaning when read in light of the specification, and may include but is not limited to, for example, a substance that can form a complex with a biomolecule. By way of example and not of limitation, ligands can include substrates, proteins, small molecules, inhibitors, activators, nucleic acids or neurotransmitters. Binding can occur through intermolecular forces, for example ionic bonds, hydrogen bonds, or van der walls interactions. Ligand binding to a receptor protein can alter the three-dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

As described in the alternatives herein, is the interlekin 13 receptor subunit alpha-2 (IL-13Rα2). “Interleukin-13 receptor subunit alpha-2 (IL-13Rα2)”, also known as CD213A2 (cluster of differentiation 213A2), has its plain and ordinary meaning when read in light of the specification, and may include but is not limited to, for example, a membrane bound protein that in humans is encoded by the IL-13RA2 gene. IL-13Rα2 is closely related to IL-13Rα1, a subunit of the interleukin-13 receptor complex. IL-13Rα2 generally binds IL-13 with high affinity, but lacks any significant cytoplasmic domain, and does not appear to function as a signal mediator. It is, however able to regulate the effects of both IL-13 and IL-4, despite the fact it is unable to bind directly to the latter. It is also reported to play a role in the internalization of IL-13.

In some alternatives, the peptide spacer is 15 amino acids or less but not less than 1 or 2 amino acids. In some embodiments, the spacer is a polypeptide chain. In some embodiments, the spacer is a polypeptide chain. In some aspects, the polypeptide chain may range in length, such as from 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. A spacer can comprise any 20 amino acids, for example, in any order to create a desirable length of polypeptide chain in a chimeric antigen receptor, which includes the amino acids arginine, histidine, lysine, aspartic acid, glutamic acid, serine, threonine, asparagine, glutamine, cysteine, glycine, proline, alanine, valine, isoleucine, methionine, phenylalanine, tyrosine and/or tryptophan. A spacer sequence can be a linker between the scFv (or ligand binding domain) and the transmembrane domain of the chimeric antigen receptor. In some alternatives of the chimeric antigen receptor, the chimeric antigen receptor further comprises a sequence encoding a spacer. In some alternatives the spacer comprises a sequence with a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. In some alternatives, the spacer resides between the scFv and the transmembrane region of the chimeric antigen receptor. In some alternatives, the spacer resides between the ligand binding domain of the chimeric antigen receptor and the transmembrane region of the chimeric antigen receptor.

A spacer may also be customized, selected, or optimized for a desired length so as to improve binding of scFv domain to the target cell, which may increase cytotoxic efficacy. In some alternatives, the linker or spacer between the scFv domain or ligand binding domain and the transmembrane can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

Some embodiments include polypeptide sequences or conservative variations thereof, such as conservative substitutions in a polypeptide sequence. In some embodiments, “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence chain | Gly, Pro |
| orientation | |
| aromatic | Trp, Tyr, Phe, His |

High levels of Interleukin 13 receptor alpha 2 (IL-13RA2) are found on a number of cancer cells including pancreatic, breast, and ovarian cancers or malignant gliomas, such as glioblastoma. IL-13RA2 has also been shown to be over-expressed in a vast majority of human patients with high-grade astrocytomas (see PLoS One. 2013 Oct. 16; 8(10):e77719; herein expressly incorporated by reference in its entirety). Additionally, previous research has shown that reducing the amount of IL13RA2 expression in cancer cells significantly slowed tumor growth in models (Breast Cancer Research, 2015; 17 (1); herein expressly incorporated by reference in its entirety). It is contemplated that few types of normal tissues express IL-13-RA2 and only at low levels.

In the case of the glioblastoma multiforme (GBM), the high expression of IL13Rα2 can be a prognostic marker of tumor progression and poor patient survival. Provided are chimeric antigen receptors (CARs), and cells and therapies containing or using the same, which selectively target IL13Rα2. The provided CARs generally comprise an extracellular domain linked to a transmembrane region and an intracellular signaling region including one or more signaling domains, generally including primary and costimulatory signaling domains.

The extracellular domain includes an antigen-binding motif, which in some aspects is or includes an antigen binding antibody fragment such as an scFv. In some aspects, the binding domain, such as the scFv, is derived from a human interleukin-13Rα2-specific antibody. In some embodiments, the CAR includes a spacer region that connects the extracellular binding domain to the transmembrane domain. In some aspects, one or more properties of the spacer are designed to optimize features of the CAR such as CAR function, for example, by providing appropriate distance between contact of the epitope and binding region and the cell membrane(s), and/or by providing flexibility. In some aspects, the spacer contains fewer or no more than 20 amino acids in length, such as fewer or no more than 15 amino acids in length, fewer or no more than 14 amino acids in length, fewer or no more than 13 amino acids in length, or no more than 12 amino acids in length. In some aspects, it contains at or about 12 or 15 amino acids in length. In some aspects, the spacer comprises all or a portion of an immunoglobulin hinge region (such as a human IgG hinge region, such as a human IgG4 hinge region) or modified version thereof.

In some alternatives, the spacer comprises a hinge region of a human antibody. In some alternatives of the method, the spacer comprises an IgG4 hinge. In some alternatives, the IgG4 hinge region is a modified IgG4 hinge. A “modified IgG4 hinge” as described herein can refer to a hinge region that can have at least 90%, 91%, 92%, 93%, 94% 95%, 96%, 97%, 98%, 99%, or 100% sequence identity or a sequence identity within a range defined by any two of the aforementioned percentages, with a hinge region amino acid sequence as set forth in SEQ ID NO:01, SEQ ID NO:02, SEQ ID NO:03, SEQ ID NO:04, SEQ ID NO:05, SEQ ID NO:06, SEQ ID NO:07, or SEQ ID NO:08.

In some alternatives, the CAR comprises an S spacer, M spacer or an L spacer. The S spacer comprises a sequence set forth in SEQ ID NO:09. The M spacer comprises a sequence set forth in SEQ ID NO:10. The L spacer comprises a sequence set forth in SEQ ID NO:11.

In some embodiments, the transmembrane portion includes or is a CD28 transmembrane domain (CD28tm) such as a human CD28 transmembrane domain. The CD28tm is encoded by a sequence set forth in SEQ ID NO:12. The CD28tm comprises the amino acids set forth in SEQ ID NO:13. In some embodiments, the transmembrane domain is linked to an intracellular signaling region containing one or more costimulatory domains, such as a costimulatory domain derived from intracellular segment of human 4-1BB (CD137) and a signaling domain of CD3-zeta (CD3 ζ) such as of human CD3ζ. In some alternatives, the CAR design is based on different combinations of two scFv molecules, which target different extracellular epitopes of the IL13Rα2, with different VH-VL orientations and three possible lengths of CAR spacer. The rest of the CAR backbone, the transmembrane and the costimulatory domains are shared between the CARs. In more alternatives, a truncated version of the epidermal growth factor receptor (EGFRt) is also included, which is co-expressed with the CARs on the T cell surface. Additional alternatives may include a dihydrofolate reductase double mutant (DHFRdm) transgene, which allows methotrexate selection of T-cell products. As described herein, both CD8 and CD4 IL13Rα2 CAR-T cells exhibit potent in vitro tumor cytotoxicity and specific efficacy in vivo against glioblastoma tumor models.

Some alternatives include a CAR containing an scFv including VL and VH domains of an antibody referred to as IL13Rα2 Ab01, in the VL-linker-VH orientation. In some aspects, such CARs further include particular combination(s) of spacer, transmembrane and/or signaling domains. For example, in some aspects, such CARs include a spacer consisting or consisting essentially of no more than 15 or no more than 12 amino acids and/or containing or consisting or consisting essentially of an immunoglobulin hinge region or modified variant thereof, such as a hinge region of an IgG4 or modified variant thereof. In some aspects, the CAR domains further include a costimulatory and a primary signaling domain, such as 41BB and CD3zeta signaling domains. In some aspects, preferred alternatives may be advantageous in their ability to augment T cell effector functions, such as by leading to increased cytotoxicity and cytokine secretion in vitro and/or improving function in vivo, such as in the context of human glioblastoma, as compared to alternative CARs, such as those containing different binding domains (e.g., with different VH and/or VL regions and/or in which the VH and VL regions are present in a different orientation) and/or those containing the same binding domain but with one or more differences in other region(s) of the CAR, such as with an alternative spacer, such as a spacer with an increased length. Embodiments provided herein, in some aspects, are based on observations herein demonstrating ability to control tumor growth and/or increase median survival of subjects bearing IL-13Ra2-expressing tumors, such as in a mouse model involving mice bearing intracranially-engrafted human GBM, treated with a single injection of the IL13Rα2 targeted CART-cells.

### Certain Nucleic Acids

Some embodiments of the methods and compositions provided herein include nucleic acids encoding a chimeric antigen receptor, the chimeric antigen receptor comprising: a) a ligand binding domain that binds to and/or interacts with an IL-13 alpha 2 (IL13Rα2) receptor and/or that comprises a CDR3, and optionally a CDR1 and a CDR2, of a VH region of SEQ ID NO:18 and a CDR3, and optionally a CDR1 and a CDR2, of a VL region of SEQ ID NO:19, b) a polypeptide spacer between the ligand binding domain and a transmembrane domain; c) the transmembrane domain; and d) intracellular signaling region. In some alternatives, the ligand binding domain is an antibody fragment, such as an antigen-binding fragment. In some alternatives, the ligand binding domain is single chain variable fragment (scFv). In some alternatives, the spacer is 15 amino acids or less but not less than 1 or 2 amino acids. In some alternatives, the spacer comprises an amino acid sequence of X₁PPX₂P. In some alternatives, the spacer region comprises a portion of a hinge region of a human antibody or modified variant thereof. In some alternatives, the intracellular signaling region comprises primary and a costimulatory signaling domains, optionally comprising all or a portion of a CD3 zeta in combination with a co-stimulatory domain selected from the group consisting of signaling domains of CD27, CD28, 4-1BB, OX-40, CD30, CD40, PD-1, ICOS, LFA-1, CD2, CD7, NKG2C, and B7-H3 and combinations thereof. In some alternatives, the intracellular signaling region comprises a signaling portion of a CD3 zeta and a signaling portion of a 4-1BB. In some alternatives, the transmembrane domain comprises a CD28 transmembrane domain (CD28tm). In some alternatives, the spacer comprises, consists of, or consists essentially of, an IgG4 hinge spacer (S). In some alternatives, the spacer comprises, consists of, or consists essentially of, an IgG4 hinge-CH3 spacer (M). In some alternatives, the spacer comprises, consists of, or consists essentially of, an IgG4 hinge-CH2 (L234D, N297A)-CHE spacer (L). In some alternatives, the scFv comprises a VL-VH orientation and spacer S. In some alternatives, scFv comprises a VH-VL orientation and a spacer, wherien the spacer is Spacer M or Spacer L. In some alternatives, the single chain variable fragment (scFv) comprises a sequence set forth in SEQ ID NO:61 or SEQ ID NO:62. In some alternatives, the single chain variable fragment (scFv) comprises a sequence set forth in SEQ ID NO:63 or SEQ ID NO:64. In some alternatives, the nucleic acid further comprises a nucleic acid that encodes a marker sequence. In some alternatives, the marker sequence is a truncated receptor and optionally is an EGFRt, a HER2t, or a CD19t. In some alternatives, the nucleic acid further comprises a dihydrofolate reductase transgene configured for methotrexate selection. In some alternatives, the dihydrofolate reductase transgene is a dihydrofolate reductase double mutant (DHFRdm). In some alternatives, the first, second, third, and/or fourth polynucleotide is codon optimized to reduce the total GC/AT ratio of the nucleic acid. In some alternatives, the first, second, third, and/or fourth polynucleotide is codon optimized for expression in humans. In some alternatives, the dihydrofolate reductase double mutant comprises amino acid mutations of L22F and F31S.

Some embodiments of the methods and compositions provided herein include expression vectors comprising the nucleic acid of any one of the alternatives herein. In some alternatives, the vector is a viral vector. In some alternatives, the vector is a lentiviral or adenoviral vector.

### Certain Chimeric Receptors

Some embodiments of the methods and compositions provided herein include chimeric receptor polypeptides, encoded by the nucleic acid of any one of the alternatives herein or by the vector of any one of the alternatives herein.

### Certain Host Cells

Some embodiments of the methods and compositions provided herein include host cells comprising the nucleic acid of any one of the alternatives herein or the expression vector of any one of the alternatives herein or expresses the chimeric receptor of any one of the alternatives herein. In some embodiments, a host cell comprises a genetically modified cell. In some alternatives, the chimeric antigen receptor is encoded by the nucleic acid of any one of the alternatives herein or by the vector of any one of the alternatives herein. In some alternatives, the expression vector comprises the nucleic acid of any one of the alternatives herein is provided. In some alternatives, the host cell is a CD8+ T cytotoxic lymphocyte cell selected from the group consisting of naïve CD8+ T cells, central memory CD8+ T cells, effector memory CD8+ T cells and bulk CD8+ T cells. In some alternatives, the CD8+ cytotoxic T lymphocyte cell is a central memory T cell and, wherein the central memory T cell is positive for CD45RO+, CD62L+, and CD8+. In some alternatives, the host cell is a CD4+ T helper lymphocyte cell selected from the group consisting of naïve CD4+ T cells, central memory CD4+ T cells, effector memory CD4+ T cells, and bulk CD4+ T cells. In some alternatives, the CD4+ helper lymphocyte cell is a naïve CD4+ T cell and, wherein the naïve CD4+ T cell is positive for CD45RA+, CD62L+ and CD4+ and negative for CD45RO. In some alternatives, the host cell is a precursor T cell. In some alternatives, the host cell is a hematopoietic stem cell.

Some embodiments of the methods and compositions provided herein include a host cell of any one of the alternatives herein or the composition of any one of the alternatives herein for use in the treatment or inhibition of a cancer or a solid tumor expressing an IL-13α2 receptor is provided. The composition comprises the host cell of any one of the alternatives herein and a pharmaceutical excipient. In some alternatives, the cancer is a glioblastoma tumor. In some alternatives, the cancer is glioblastoma multiforme (GBM). In some alternatives, the cancer is an IL13Rα-positive malignancy. In some alternatives, the cancer is brain cancer or brain tumors.

### Certain Compositions

Some embodiments of the methods and compositions provided herein include compositions comprising a host cell of any one of the alternatives herein, and a pharmaceutically acceptable excipient.

### Certain Methods of Preparing Host Cells

Some embodiments of the methods and compositions provided herein include methods for preparing a host cell of any one of the alternatives herein is provided comprising: a) introducing a nucleic acid of any one of the alternatives herein or an expression vector of any one of the alternatives herein into lymphocytes; and b) culturing the lymphocytes in the presence of anti-CD3 and/or anti CD28 antibodies, and at least one homeostatic cytokine until the cells expand; and c) selecting the lymphocytes with a selection reagent; wherein the selection reagent is configured to selectively enrich cells transduced with the nucleic acid or vector. In some alternatives, the selection reagent is methotrexate. In some alternatives, the lymphocytes have a CD45RA−, CD45RO+, and CD62L+ phenotype. In some alternatives, the lymphocytes are CD8+ or CD4+. In some alternatives, the cytokine is IL-15, 11-7 and/or 11-21. In some alternatives, the method further comprises introducing a second nucleic acid into the host cell, the second nucleic acid encoding a marker protein. In some alternatives, the marker protein is EGFRt.

### Certain Methods of Therapy

Some embodiments of the methods and compositions provided herein include methods of performing cellular immunotherapy in a subject having a cancer or a tumor comprising: administering the host cell of anyone of the alternatives herein or the composition of the alternatives herein is provided to the subject. The composition comprises a host cell of any one of the alternatives herein, and a pharmaceutically acceptable excipient is provided. In some alternatives, the cancer is glioblastoma tumor. In some alternatives, the cancer is glioblastoma multiforme (GBM). In some alternatives, the cancer is an IL13Rα-positive malignancy. In some alternatives, the cancer is brain cancer. In some alternatives, the subject is selected to receive combination therapy. In some alternatives, the combination therapy comprises administering a chemotherapeutic drug. In some alternatives, the combination therapy comprises administering radiation therapy. In some alternatives, the chemotherapeutic drug comprises electochemotherapy, alkylating agents, antimetabolites (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine), anti-tumor antibiotics, topoisomerase inhibitors, mitotic inhibitors, corticosteroids, DNA intercalating agents, or checkpoint inhibitors (checkpoint kinases CHK1, CHK2). In some alternatives, the cancer is a glioma.

### Development of Certain IL13Ra2 CARs

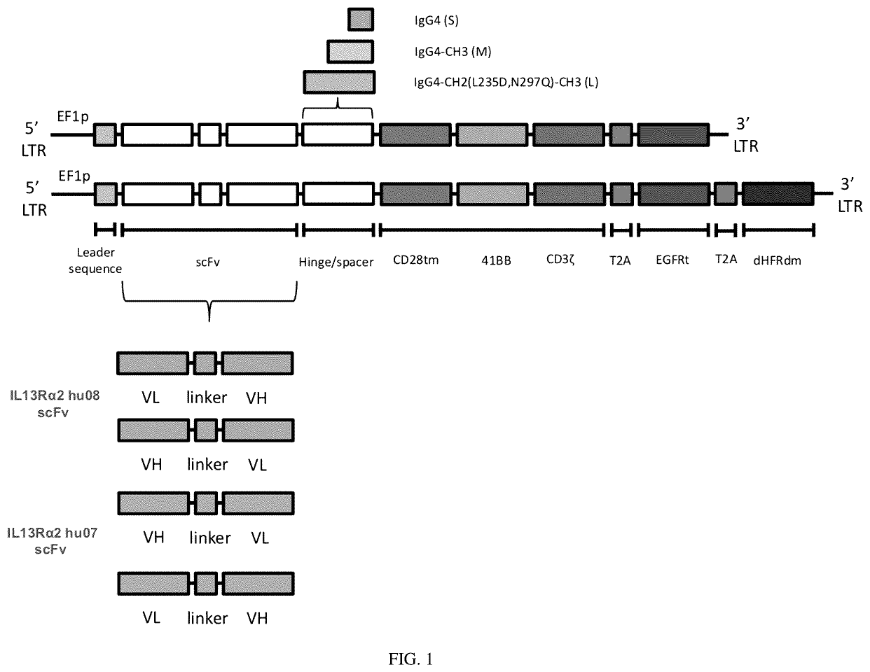

Shown in FIG. 1 are schematic representations of the nucleic acids encoding the structure of several IL-13Rα2 CARs that were created, each having one of various binding domains, one of various spacer regions, together with a transmembrane domain and intracellular signaling domain(s). The nucleic acid sequence of each of these exemplary IL-13R13Rα2 CARs includes a leader sequence, which may be used for example to allow translation of the mRNA transcript encoding the CAR. Each of the exemplary CARs also comprised an IL-13Rα2 scFv, specifically an anti-IL13Rα2 scFv having VH and VL domains set forth in SEQ ID NOs:14 and 16, respectively or having VH and VL domains set forth in SEQ ID NOs:15 and 17, respectively. Each of the CARs generated and tested were generated with four different scFv binding domains. The four binding domains included each of the anti-IL13Rα2 VH/VL pairs, in each of the VL-VH and VH-VL orientation, individually.

The three different spacers, individually present in the exemplary CARs, are shown in FIG. 1, positioned between the scFv and the transmembrane domain. The spacers tested varied in length and were derived from immunoglobulin constant regions. The spacer with the shortest amino acid sequence length was a modified human IgG4 hinge region), a spacer with an intermediate (medium) length included the modified human hinge region and further included a CH3 domain (IgG4-CH3); the longest spacer tested included the modified IgG4 hinge region, a modified CH2 region and the CH3 region (IgG4-CH2 (L235D, N297Q)-CH3 (L)). Each of the exemplary CARs generated further included a transmembrane derived from human CD28 (CD28tm domain) and signaling domains of human 4-1BB and CD3zeta (4-1BB domain, CD3 zeta domain).

Each of the nucleic acids encoding and used to express the CARs further included a sequence encoding a T2A skip sequence and a nucleic acid encoding a truncated surrogate marker of CAR expression.

In some alternatives, the ligand binding domain of embodiments of the provided CARs comprises a heavy chain variable region that has or consists of the amino acid sequence of SEQ ID NO:18, or has or consists of a sequence having at least 90, 91, 92, 93, 94, 95, 96, 97, 98, or 99% identity to SEQ ID NO:18, or that comprises a CDR1, CDR2, and/or CDR3 of, typically at least a CDR3 of, a VH region having the sequence of SEQ ID NO:18.

In some alternatives, the ligand binding domain of embodiments of the provided CARs comprises a light chain variable region that has or consists of the amino acid sequence of SEQ ID NO:19, or has or consists of a sequence having at least 90, 91, 92, 93, 94, 95, 96, 97, 98, or 99% identity to SEQ ID NO:19, or that comprises a CDR1, CDR2, and CDR3 of a VL region having the sequence of SEQ ID NO:19. Sequences have been described in WO2014072888, included by reference in its entirety herein.

In some alternatives, the ligand binding region that specifically binds to human IL-13-Ra2 comprises: (a) a heavy chain CDR1 comprising SEQ ID NO:20; (b) a heavy chain CDR2 comprising SEQ ID NO:21; (c) a heavy chain CDR3 comprising SEQ ID NO:22; and/or comprises (d) a light chain CDR1 comprising SEQ ID NO:23; (e) a light chain CDR2 comprising SEQ ID NO:24; and/or (f) a light chain CDR3 comprising SEQ ID NO:25.

In some alternatives, the ligand binding region comprises the heavy chain variable region amino acid sequence of SEQ ID NO:18 and the light chain variable region amino acid sequence of SEQ ID NO:19.

In some alternatives, the ligand binding domain comprises a heavy chain CDR2 (CDRH2) comprising SEQ ID NO:28; (c) a heavy chain CDR3 (CDRH3) comprising SEQ ID NO:29; (d) a light chain CDR1 comprising SEQ ID NO:30; (e) a light chain CDR2 comprising SEQ ID NO:31; and/or (f) a light chain CDR3 comprising SEQ ID NO:32.

In some alternatives, the ligand binding domain comprises a heavy chain CDR1 (CDR-H1) comprising a sequence set forth in SEQ ID NO:33, SEQ ID NO:34, SEQ ID NO:35, or SEQ ID NO:36. In some alternatives CDR-H2 comprises a sequence set forth in SEQ ID NO:37, SEQ ID NO:38, SEQ ID NO:39, or SEQ ID NO:40. In some alternatives, CDRH3 comprises a sequence set forth in SEQ ID NO:41.

In some embodiments, the CAR comprises a spacer interposed between the binding domain and transmembrane domain. In some alternatives, the spacer comprises or consists of or consists essentially of at least a portion of an immunoglobulin constant region, such as all or a portion of an immunoglobulin hinge region, such as an IgG4 hinge region, or a functional variant thereof. In some embodiments, the immunoglobulin constant and/or hinge region is a region of human IgG, such as IgG4 or IgG1, or a variant thereof. The spacer can be of a length that provides for increased responsiveness of the cell following antigen binding, as compared to in the absence of the spacer. Exemplary spacers include those described in international patent application publication number WO2014031687, hereby expressly incorporated by reference in its entirety. In some examples, the spacer is or is about 12 amino acids in length or is no more than 12 amino acids in length or is or is about 15 amino acids in length or is or is about no more than 15 amino acids in length.

Example spacers include those having at least 10 to 229 amino acids, 10 to 200 amino acids, 10 to 175 amino acids, 10 to 150 amino acids, 10 to 125 amino acids, 10 to 100 amino acids, 10 to 75 amino acids, 10 to 50 amino acids, 10 to 40 amino acids, 10 to 30 amino acids, 10 to 20 amino acids, or 10 to 15 amino acids, and including any integer between the endpoints of any of the listed ranges. In some embodiments, a spacer region has 12 amino acids or less but not zero, 119 amino acids or less but not zero, or 229 amino acids or less but not zero. In some embodiments, the spacer is less than 250 amino acids in length but not zero, less than 200 amino acids in length but not zero, less than 150 amino acids in length but not zero, less than 100 amino acids in length but not zero, less than 75 amino acids in length but not zero, less than 50 amino acids in length but not zero, less than 25 amino acids in length but not zero, less than 20 amino acids in length but not zero, less than 15 amino acids in length but not zero, less than 12 amino acids in length but not zero, or less than 10 amino acids in length but not zero. In some embodiments, the spacer is from or from 10 to 250 amino acids in length, 10 to 150 amino acids in length, 10 to 100 amino acids in length, 10 to 50 amino acids in length, 10 to 25 amino acids in length, 10 to 15 amino acids in length, 15 to 250 amino acids in length, 15 to 150 amino acids in length, 15 to 100 amino acids in length, 15 to 50 amino acids in length, 15 to 25 amino acids in length, 25 to 250 amino acids in length, 25 to 100 amino acids in length, 25 to 50 amino acids in length, 50 to 250 amino acids in length, 50 to 150 amino acids in length, 50 to 100 amino acids in length, 100 to 250 amino acids in length, 100 to 150 amino acids in length, or 150 to 250 amino acids in length. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. Clin. Cancer Res., 19:3153 (2013), international patent application publication number WO2014031687, U.S. Pat. No. 8,822,647 or published app. No. US2014/0271635 hereby expressly incorporated by reference in their entireties.

In some aspects, the spacer of the CAR consists of or comprises an IgG4 hinge, an IgG4 hinge-CH3 or an IgG4 hinge-CH2(L235D, N297Q)-CH3.

In some alternatives, the ligand binding domain comprises (a) a heavy chain variable region comprising a CDR1, a CDR2, and a CDR3 of the VH sequence of SEQ ID NO:18; and, (b) a light chain variable region comprising a CDR1, a CDR2, and a CDR3 of the VL sequence of SEQ ID NO:19. Sequences can be found in WO2014/072888, which is hereby expressly incoporated by reference in its entirety herein.

In some alternatives, the orientation of the chains is VL-linker-VH. In some aspects, the orientation is VH-linker VL.

In some alternatives, the ligand binding region comprises a heavy chain variable region comprising a CDR1, CDR2, and CDR3 of the VH sequence shown in SEQ ID NO:18; a light chain variable region comprising a CDR1, CDR2, and CDR3 of the VL sequence shown in SEQ ID NO:19. In some alternatives, a spacer is provided wherein the spacer comprises an immunoglobulin constant region, such as one including all or part of an Fc region that includes at least one pair of amino acid substitutions selected from the group consisting of the amino acid sequence of SEQ ID NO:42 and SEQ ID NO:43; or an engineered Fc region and at least one engineered light chain constant region selected from group consisting of L443C (SEQ ID NO:44), Q347C (SEQ ID NO:45), kK183C (SEQ ID NO:46), SEQ ID NO:47, L443C/kK183C (SEQ ID NO:44 and SEQ ID NO:46), Q347C/kA111 C (SEQ ID NO:45 and SEQ ID NO:47), and Q347C/kK183C (SEQ ID NO:45 and SEQ ID NO:46).

In some alternatives, the constant region or portion thereof comprises a sequence set forth in SEQ ID NO:48.

In some alternatives, Ab02-derived scFv comprises a sequence set forth in SEQ ID NO:49 (LCDR1), SEQ ID NO:50 (LCDR2) and/or SEQ ID NO:51 (LCDR3). In some alternatives, the Ab02 antibody comprises a sequence as set forth in SEQ ID NO:55 (HCDR1), SEQ ID NO:56 (HCDR2), and/or SEQ ID NO:57 (HCDR3).

In some alternatives, the Ab01 antibody comprises a sequence set forth in SEQ ID NO:52 (LCDR1), SEQ ID NO:53 (LCDR2) and/or SEQ ID NO:54 (LCDR3). In some alternatives, the Ab01 antibody comprises a sequence as set forth in SEQ ID NO:58 (HCDR1), SEQ ID NO:59 (HCDR2), and/or SEQ ID NO:60 (HCDR3).

In some embodiments, the CAR includes an antigen-binding region that is or comprises an scFv including VH and VL domains in the VL-linker-VH orientation. In some embodiments, such as in some aspects of such embodiments, the VH domain comprises an amino acid sequence of SEQ ID NO:18, a CDR3 (and, optionally, CDRs 1 and 2) of the VH sequence set forth in SEQ ID NO:18, and/or comprises or consists of an amino acid sequence having at least 90, 91, 92, 93, 94, 95, 96, 97, 98, or 99% identity to SEQ ID NO:18, or that comprises a CDR1, CDR2, and/or CDR3 of, typically at least a CDR3 of, a VH region having the sequence of SEQ ID NO:18; in some aspects, e.g., of such embodiments, the VH is a VH derived from Ab01. In some aspects of such embodiments, the VL domain comprises an amino acid sequence of SEQ ID NO:19, a CDR3 (and, optionally, CDRs 1 and 2) of the VL sequence set forth in SEQ ID NO:19, and/or comprises or consists of an amino acid sequence having at least 90, 91, 92, 93, 94, 95, 96, 97, 98, or 99% identity to SEQ ID NO:19, or that comprises a CDR1, CDR2, and/or CDR3 of, typically at least a CDR3 of, a VL region having the sequence of SEQ ID NO:19 in some aspects, e.g., of such embodiments, the VH is a VH derived from Ab01. In some aspects of any of such embodiments, such as aspects of the CARs having such binding domains, the CAR includes a spacer region between the binding domain and transmembrane domain that contains no more than at or about 50, 40, 30 or 20 amino acids in length, such as no more than at or about 15 or no more than at or about 12, amino acids, and/or contains or is or consists essentially of an immunoglobulin hinge region or variant thereof, such as an IgG4 hinge or variant thereof.

In some aspects, the CARs and/or cells expressing the same exhibit one or more increased or superior functional activity as compared to a reference CAR. In some aspects, the reference CAR is a CAR having the same binding domain (and optionally otherwise identical) but having a spacer differing in length and/or composition, such as one greater than at or about 50, 40, 30, 20, 15, or 12 amino acids in length, and/or a CAR having a binding domain with the same or similar variable regions, but in an alternative orientation. In some embodiments, the function is as observed in an in vitro assay such as a co-culture assay with cells expressing IL-13Rα2 (such as an assay measuring cytokine production or cytolytic activity) or an in vivo assay using an animal model with a tumor positive for IL-13Rα2, such as one assessing tumor burden reduction or survival following administration of cells, such as T cells, expressing the CAR.

In the alternatives herein, eight CARs having various combinations of antigen-binding domains and other domains as depicted in FIG. 1 were tested in various assays.

### Certain Sequences

In some alternatives, the Ab01 VH comprises a sequence set forth in SEQ ID NO:14. In some alternatives, the Ab01 VL comprises a sequence set forth in SEQ ID NO:16. In some alternatives, the IL13Rα2 Ab01 VLVH scFv is encoded by a sequence set forth in SEQ ID NO:61. In some alternatives, the IL13Rα2 Ab01 VHVL scFv is encoded by a sequence set forth in SEQ ID NO:62 which includes an Ab01-VL sequence, an Ab01-VH sequence, an ATG start codon, a 5′ NheI restriction site (GCTAGC), and a 3′ RsrII restriction site (CGGACCG). In some alternatives, the Ab01-VH protein sequence comprises SEQ ID NO:65. In some alternatives, Ab01 HCDR1 comprise a sequence set forth in SEQ ID NO:66. In some alternatives, Ab01 HCDR2 comprise a sequence set forth in SEQ ID NO:67. In some alternatives, Ab01 HCDR3 comprise a sequence set forth in SEQ ID NO:68. In some alternatives, Ab01-VL comprise a sequence set forth in SEQ ID NO:69. In some alternatives, Ab01 LCDR1 comprise a sequence set forth in SEQ ID NO:70. In some alternatives, Ab01 LCDR2 comprise a sequence set forth in SEQ ID NO:71. In some alternatives, Ab01 LCDR3 comprise a sequence set forth in SEQ ID NO:72.

In some alternatives, the Ab02 VH comprises a sequence set forth in SEQ ID NO:15. In some alternatives, the Ab02 VL comprises a sequence set forth in SEQ ID NO:17. In some alternatives, the IL13Rα2 Ab02 VLVH scFv is encoded by a sequence set forth in SEQ ID NO:63. In some alternatives, the IL13Rα2 Ab02 VHVL scFv is encoded by a sequence set forth in SEQ ID NO:64, which includes an Ab02-VL sequence, an Ab02-VH sequence, an ATG start codon, a 5′ NheI restriction site (GCTAGC), and a 3′ RsrII restriction site (CGGACCG). In some alternatives, Ab02-VH protein sequence comprise a sequence set forth in SEQ ID NO:73. In some alternatives, Ab02 HCDR1 comprise a sequence set forth in SEQ ID NO:74. In some alternatives, Ab02 HCDR2 comprise a sequence set forth in SEQ ID NO:75. In some alternatives, Ab02 HCDR3 comprise a sequence set forth in SEQ ID NO:76. In some alternatives, Ab02-VL protein sequence comprise a sequence set forth in SEQ ID NO:77. In some alternatives, Ab02 LCDR1 comprise a sequence set forth in SEQ ID NO:78. In some alternatives, Ab02 LCDR2 comprise a sequence set forth in SEQ ID NO:79. In some alternatives, Ab02 LCDR3 comprise a sequence set forth in SEQ ID NO:80.

### Examples

### Example 1—Construction of IL-13Rα2-Targeting CARs

Various IL-13Rα2-targeting CARs were constructed. As shown in FIG. 1, IL-13Rα2 specific CARs included: one of a number of single-chain variable fragment (scFv) that specifically recognized an extracellular epitope of IL-13 alpha 2 receptor (IL-13Rα2); one of various spacer domains (marked “S”, “M” and “L”); a transmembrane domain derived from a human CD28 (CD28tm); a costimulatory domain derived from human 4-1BB; a CD3ζ-derived signaling domain; a T2A ribosomal skip sequence; and a truncated EGFR (EGFRt) transduction marker; and optionally a further T2A ribosomal skip sequence and a dihydrofolate reductase double mutant (DHFRdm) transgene configured for methotrexate selection.

Examples of antibodies or epitope binding fragments from which single-chain variable fragment (scFv) can be derived include humanized anti-IL-13-Ra2 IgG1 antibody hu08 (Creative Biolabs, NY), and Anti-IL13RA2 Therapeutic Antibody (hu07 v1.1) (Creative Biolabs, NY).

The spacer domain was linked in each CAR with the scFv and connected the extracellular binding element to the transmembrane domain. Each of the CARs was generated to include one of the three different spacers (S, M, or L), each with an amino acid sequence of a different length (Spacer S, SEQ ID NO:9; Spacer M, SEQ ID NO:10; or a Spacer L, SEQ ID NO:11). Different scFv configurations used individually in the various IL-13Rα2 CARs, including VH and VL domains derived from one of two different anti-IL-13Rα2-directed antibodies (‘Hu08’, Ab01; and ‘Hu07’, Ab02). The scFv portion of each CAR was comprised of a VH region having a sequence as set forth in SEQ ID NO:14 or SEQ ID NO:15 and a VL region having a sequence set forth in SEQ ID NO:16 or SEQ ID NO:17, present in a VL-VH or VH-VL orientation, connected by a flexible peptide linker.

### Example 2—Functional Comparison of CARs Derived from Different Antibodies

A functional comparison of CARs including scFv binding domains derived from different antibodies in different orientations was performed. The comparison included a surface expression analysis of EGFRt, and a cytokine release assay. In this study, each of the CARs included the “S” spacer (having the amino acid sequence of SEQ ID NO:9, a modified immunoglobulin hinge region), as well as the transmembrane and costimulatory and primary signaling domains described and shown in FIG. 1, with one of the various scFvs binding regions (VH/VL domains and orientations) shown in FIG. 1. CARs included: IL-13Ra2 Ab01 VLVH Spacer S CAR; IL-13Ra2 Ab01 VHVL Spacer S CAR; IL-13Ra2 Ab02 VLVH Spacer S CAR; and IL-13Ra2 Ab02 VHVL Spacer S CAR.

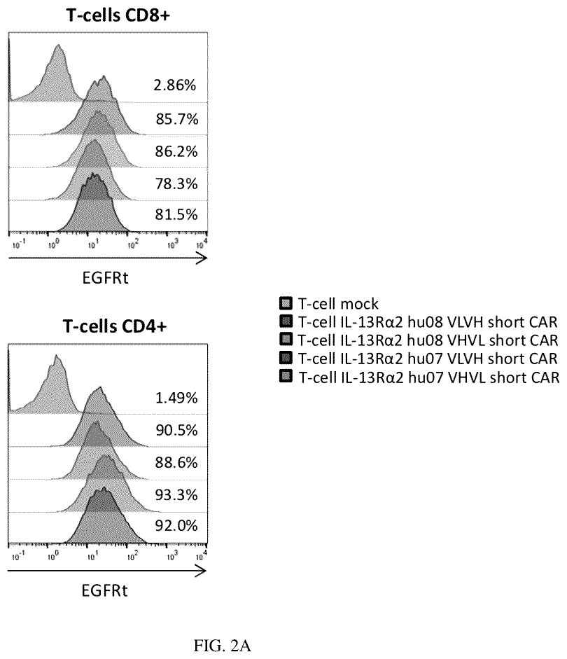

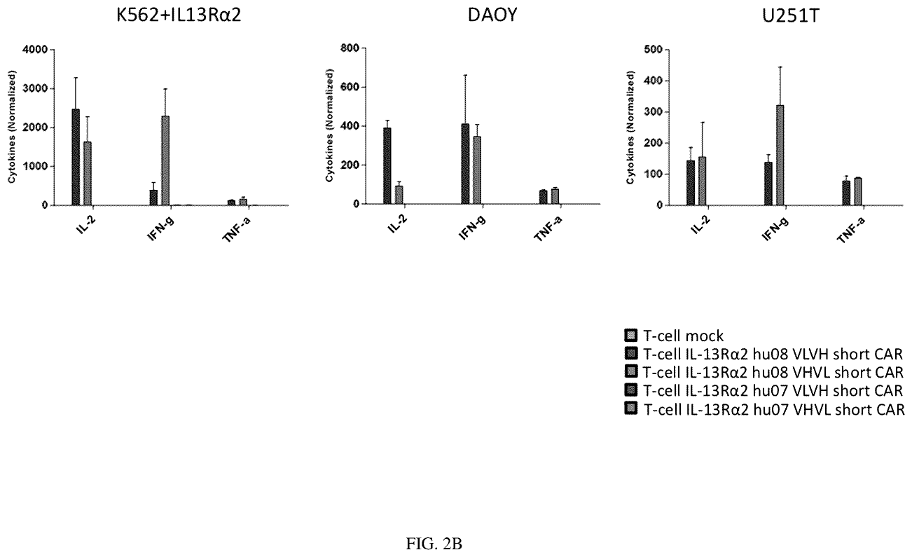

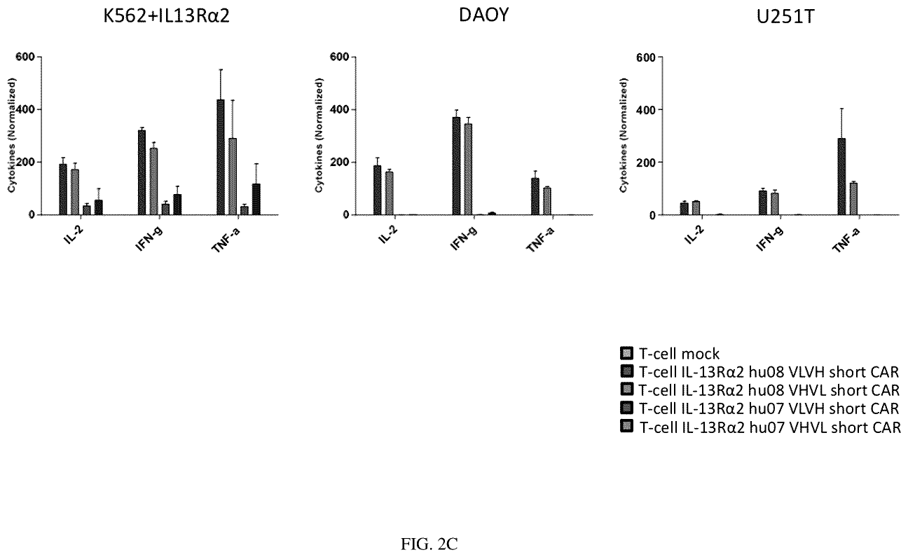

Expression of CARs in cells was examined by determining expression of the EGFRt marker in CD8+ and CD4+ T cells using flow cytometry (FIG. 2A)

CAR T cells were co-cultured, individually, with DAOY cells (human brain/cerebellum cel line), U251T cells (human giobasltoma cell line) and K562 cells (human chronic myelogenous leukemia cells) engineered to express IL13Rα2. After 24 h levels of IL-2, IFN-γ and TNF-α were measured in supernatants using a cytokine assay. Increased cytokine production following incubation with target cells was observed for CD8+ and CD4+ T cells expressing CARs containing binding domains having VH and VL domains from the Ab01 antibody, in both VH/VL orientations (FIG. 2B and FIG. 2C).

### Example 3—Functional Comparison of CARs Including Different Spacers

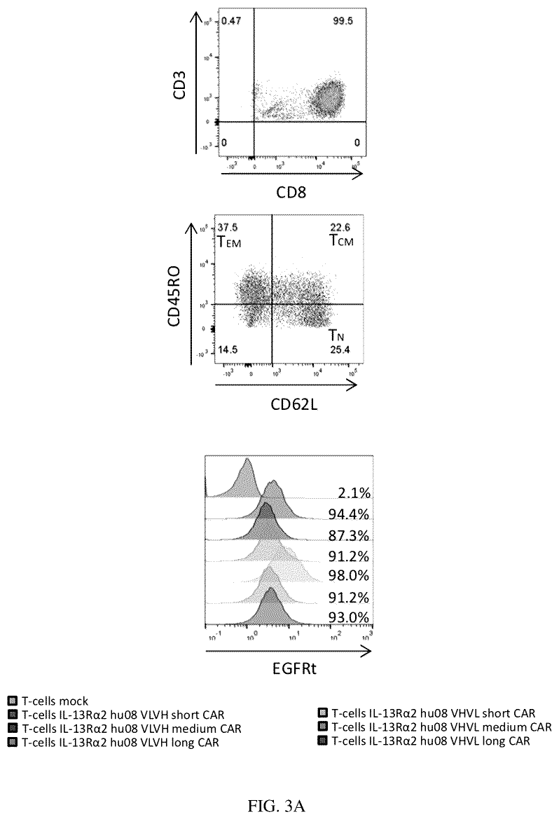

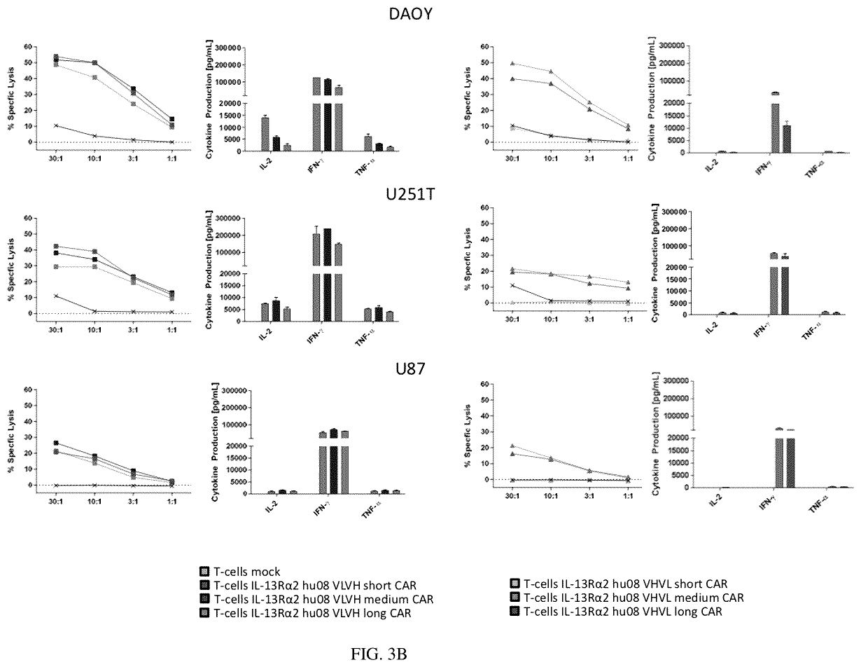

A functional comparison of CARs including scFvs derived from the same antibody (in different orientations) and different spacers of different length was performed. The four CARS depicted in FIG. 1 were tested for cell surface expression in CD8+ by gating for the marker sequence, EGFRt (FIG. 3A). Furthermore, the CD8+ cells carrying the CARs were also tested for their ability to cause specific lysis, as well as their ability to release cytokines. In this study, each of the exemplary CARs included one of two different binding domains—an scFv having VL and VH domains from Ab01, either in the VL-VH or the VH-VL orientation—and one of the various spacers (“S” (having the amino acid sequence of SEQ ID NO:9 “M” (having the amino acid sequence of SEQ ID NO:10 and “L” (having the amino acid sequence of SEQ ID NO:11), as well as the transmembrane and costimulatory and primary signaling domains described and depicted in FIG. 1.

Representative results from multi-parameter flow cytometry analysis of T cells derived from healthy donor and transduced with the indicated CARs lentivirus are showin in FIG. 3A (top and middle panels) in which histograms quadrants were drawn based on control staining. Surface surrogate marker (EGFRt) expression on CD8+IL-13Rα2 CAR T-cells was analyzed by flow cytometry (FIG. 3A, lower panle).

IL-13Rα2-targeted CAR expressing CD8 T cells were co-cultured with different IL-13Ra2-expressing target cell lines at variable indicated effector to target ratios (FIG. 3B). A chromium release assay was used to assess cytolytic activity and cytokine production was assessed in supernatants. The chromium release assay was over a 4 hr co-culture period, while cytokine release was assessed following 24 hr co-culture (2:1 effector-target ratio). As shown in FIG. 3C, co-culture with the various CAR-expressing T cells led to specific lysis, increasingly with increased effector to target ratios, and levels of cytokine secretion. Compared to other CARs assessed, increased cytolytic activity was observed for cells expressing the CAR with the “S” spacer and including the scFv with the VL-VH orientation. In each of the CARs assessed having binding domains with the VL-VH orientation, target-specific cytolytic activity and cytokine production were observed. Additionally, in CARs with the VL-VH orientation, the presence of the “S” spacer was observed to result in superior effects compared to the other spacers tested. In contrast, cytolytic activity was not observed in this assay for CARs with the VH-VL orientation and the “S” spacer (whereas activity was observed for each of the VH-VL CARs having the “M” and “L” spacers).

With regard to cytokine production, as shown in FIG. 3B, T cell with IL-13Ra2 Ab01 VLVH Spacer S CAR, T cell with IL-13Ra2 Ab01 VLVH Spacer M CAR, T cell with IL-13Ra2 Ab01 VLVH Spacer L CAR led to higher expression of IFN-gamma. T cell with IL-13Ra2 Ab01 VHVL Spacer M CAR, and T cell with IL-13Ra2 Ab01 VHVL Spacer L CAR showed IFN-gamma expression. T cell with IL-13Ra2 Ab01 VLVH Spacer S CAR, T cell with IL-13Ra2 Ab01 VLVH Spacer M CAR, T cell with IL-13Ra2 Ab01 VLVH Spacer L CAR led to higher specific lysis.

With regard to specific lysis, as shown in FIG. 3B, at increased concentrations, the CD8+ cells carrying the IL-13Rα2 Ab01 VLVH spacer S CAR, IL-13Rα2 Ab01 VLVH medium CAR, and IL-13Rα2 Ab01 VLVH long CAR caused a significant amount of specific lysis at high concentrations of cell. Furthermore, these cells led to high production of the cytokine IFN-gamma.

In contrast, cells expressing the IL-13Rα2 Ab01 VHVL spacer S CAR, IL-13Rα2 Ab01 VHVL medium CAR and IL-13Rα2 Ab01 VHVL long CAR had decreased amounts of specific lysis when compared to the IL-13Rα2 Ab01 VLVH spacer S CAR, IL-13Rα2 Ab01 VLVH medium CAR and IL-13Rα2 Ab01 VLVH long CAR. Specific lysis was not seen with the IL-13Rα2 Ab01 VHVL spacer S CAR. Additionally, IL-13Rα2 Ab01 VHVL spacer S CAR expression did not lead to IFN-gamma expression, which was seen with the IL-13Rα2 Ab01 VHVL medium CAR and the IL-13Rα2 Ab01 VHVL long CAR.

### Example 4—In Vivo Anti-Tumor Activity of an Anti-IL13Rα2 CAR

Therapeutic outcomes following administration of cells expressing an IL13Rα2-specific CAR containing a binding domain derived from antibody Ab01, VLVH orientation, spacer “S”, were evaluated in a mouse glioblastoma model. The CARs used for the experiments were expressed in CD8+ cells. For the experiments, ffLuc+ U87 glioblastoma cells (0.2×10⁶) were intracranially injected into the forebrain of NSG mice at day 0. On day 7, mice (n=3 per group) received no treatment (vehicle control or mock) or they received treatment with different doses (2×10⁶, 1×10⁶ or 0.5×10⁶ cells) of IL-13Rα2 Ab01 VLVH transduced CD8+ CAR T-cells.

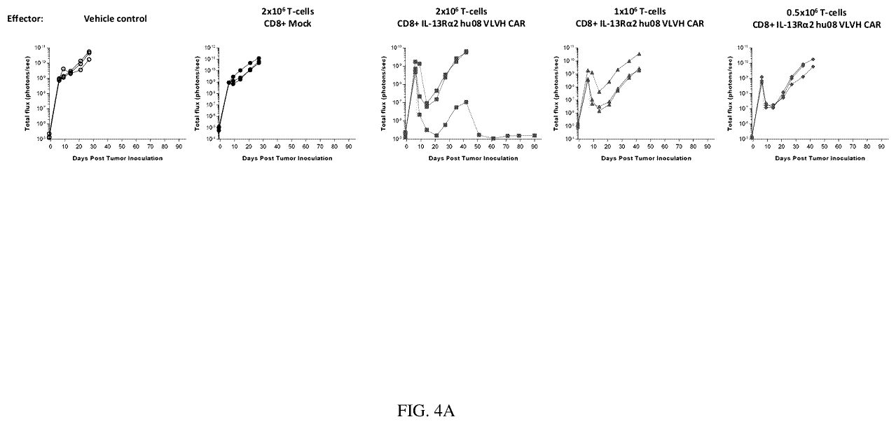

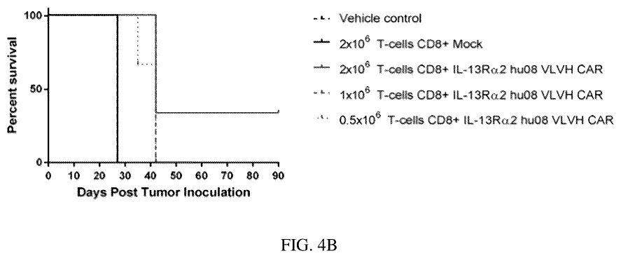

FIG. 4A and FIG. 4B show results from a study demonstrating in vivo anti-tumor activity of an IL13Rα2-specific CAR containing the VH and VL domains derived from Ab01, in the VL-VH orientation, and the “S” spacer, along with the transmembrane and signaling domains as depicted in FIG. 1. Human glioblastoma cells expressing firefly luciferase (ffLuc⁺ U87 glioblastoma cells) (0.2×10⁶) were intracranially injected into forebrains of NSG mice at day 0. On day 7, mice (n=3 per group) received Vehicle control or Mock administration or were administered treatment with different doses (2×10⁶, 1×10⁶ or 0.5×10⁶ cells) of CD8+ cells expressing the IL-13Rα2-specific CAR (Ab01 VL-VH). FIG. 4A shows plots of total flux (photons/sec) as a measure of tumor burden (y-axis) over time (days post-tumor inoculation, x-axis), for animals treated with vehicle or mock treatment (left) and indicated decreasing numbers of CAR+ cells (right-hand; right to left), demonstrating tumor regression following administration of cells expressing the CAR. A Kaplan-Meier survival curve demonstrated increasingly improved survival for mice treated with increasing doses of T cells expressing the anti-IL-13Rα2 CAR (FIG. 4B). The vehicle control had 0% survival rate at 25 days. However, mice that received 2×10⁶ of the CD8+ T-cells expressing the IL13Rα2 Ab01 VLVH had ˜50% survival after 90 days.

### Example 5—In Vivo Anti-Tumor Activity of an Anti-IL13Rα2 CAR

To assess the impact of the IL13Rα2 Ab01 VLVH CAR spacer, a experiment substantially similar to Example 3 was performed using U251T tumor-bearing mice as glioblastoma model. On day 0, U251T GFP:ffluc tumor cells were injected intracranially followed by an intratumoral CD8+ CAR T-cell or Cd8+ Mock T-cells injection on day 7. Mice injected with Mock T-cells served as control.

All mice treated with IL13Rα2 Ab01 VLVH 2G short CAR T-cells showed compete tumor regression. Only one of the CAR-medium group of mice exhibited tumor relapse and necessitated animal euthanasia at day 58 after tumor injection. Three of the U251T tumor-bearing mice treated with intratumoral injection of long spacer IL13Rα2 CAR T-cells exhibited less therapeutic activity and tumor relapse.

Taken together, all these data suggest that the extracellular short spacer size is the optimal configuration for the IL13Rα2 Ab01 VLVH conferring an optimal in vivo antitumor potency, supporting its clinical development.

TABLE 2 lists certain amino acid and nucleotide sequences for some embodiments of the methods and compositions provided herein.

TABLE 2

| SEQ ID | | |
|---|---|---|
| NO: | Feature(s) | Sequence |
| SEQ ID | Human IgG1 | EPKSCDKTHTCPPCP |
| NO: 01 | | |
| SEQ ID | Human IgG2 | ERKCCVECPPCP |
| NO: 02 | | |
| SEQ ID | Human IgG3 | ELKTPLGDTHTCPRCPEPKSCDTPPPCPRCP |
| NO: 03 | | EPKSCDTPPPCPRCPEPKSCDTPPPCPRCP |
| SEQ ID | Human IgG4 | ESKYGPPCPSCP |
| NO: 04 | | |
| SEQ ID | Modified | ESKYGPPCPPCP |
| NO: 05 | Human IgG4 | |
| SEQ ID | Modified | YGPPCPPCP |
| NO: 06 | Human IgG4 | |
| SEQ ID | Modified | KYGPPCPPCP |
| NO: 07 | Human IgG4 | |
| SEQ ID | Modified | EVVKYGPPCPPCP |
| NO: 08 | Human IgG4 | |
| SEQ ID | S spacer | ESKYGPPCPPCP |
| NO: 09 | | |
| SEQ ID | M spacer | ESKYGPPCPPCPGQPREPQVYTLPPSQEEMTKNQVSLT |
| NO: 10 | | CLVKGFYPSDIAVEWESNGQPENNYKTTPPVLDSDGSF |
| | | FLYSRLTVDKSRWQEGNVFSCSVMHEALHNHYTQKSLS |
| | | LSLGK |
| SEQ ID | L spacer | ESKYGPPCPPCPAPEFLGGPSVFLFPPKPKDTLMISRTP |
| NO: 11 | | EVTCVVVDVSQEDPEVQFNWYVDGVEVHNAKTKPREEQF |
| | | NSTYRVVSVLTVLHQDWLNGKEYKCKVSNKGLPSSIEKT |
| | | ISKAKGQPREPQVYTLPPSQEEMTKNQVSLTCLVKGFYP |
| | | SDIAVEWESNGQPENNYKTTPPVLDSDGSFFLYSRLTVD |
| | | KSRWQEGNVFSCSVMHEALHNHYTQKSLSLSLGK |
| SEQ ID | CD28tm | ATGTTCTGGGTGCTGGTGGTGGTCGGAGGC |
| NO: 12 | | GTGCTGGCCTGCTACAGCCTGCTGGTCACCGTGGCCTT |
| | | CATCATCTTTTGGGTG |
| SEQ ID | CD28tm | MFWVLVVVGGVLACYSLLVTVAFIIFWV |
| NO: 13 | | |
| SEQ ID | Ab01-VH | EVQLVESGGGLVQPGGSLRLSCAASGFTFSRNGMSWVR |
| NO: 14 | | QAPGKGLEWVATVSSGGSYIYYADSVKGRFTISRDNAK |
| | | NSLYLQMNSLRAEDTAVYYCARQGTTALATRFFDVWGQ |
| | | GTLVTVSS |
| SEQ ID | Ab02-VH | EVQLVESGGGLVQPGGSLRLSCAASGFTFTKYGVHWV |
| NO: 15 | | RQAPGKGLEWVAVKWAGGSTDYNSALMSRFTISRDNA |
| | | KNSLYLQMNSLRAEDTAVYYCARDHRDAMDYWGQGTL |
| | | VTVSS |
| SEQ ID | Ab01-VL | DIQMTQSPSSLSASVGDRVTITCKASQDVGTAVAWYQQK |
| NO: 16 | | PGKAPKLLIYSASYRSTGVPSRFSGSGSGTDFTLTISSL |
| | | QPEDFATYYCQHHYSAPWTFGGGTKVEIK |
| SEQ ID | Ab02-VL | DIQMTQSPSSLSASVGDRVTITCTASLSVSSTYLHWYQ |
| NO: 17 | | QKPGKAPKLLIYSTSNLASGVPSRFSGSGSGTDFTLTI |
| | | SSLQPEDFATYYCHQYHRSPLTFGGGTKVEIK |
| SEQ ID | VH region | EVQLVESGGGLVQPGGSLRLSCAASGFTFSRNGMSWVR |
| NO: 18 | | QAPGKGLEWVATVSSGGSYIYYADSVKGRFTISRDNAK |
| | | NSLYLQMNSLRAEDTAVYYCARQGTTALATRFFDVWGQ |
| | | GTLVTVSS |
| SEQ ID | VL region | DIQMTQSPSSLSASVGDRVTITCKASQDVGTAVAWYQQK |
| NO: 19 | | PGKAPKLLIYSASYRSTGVPSRFSGSGSGTDFTLTISSL |
| | | QPEDFATYYCQHHYSAPWTFGGGTKVEIK |
| SEQ ID | heavy chain | SRNGMS |
| NO: 20 | CDR1 | |
| SEQ ID | heavy chain | TVSSGGSYIYYADSVKG |
| NO: 21 | CDR2 | |
| SEQ ID | heavy chain | QGTTALATRFFDV |
| NO: 22 | CDR3 | |
| SEQ ID | light chain | KASQDVGTAVA |
| NO: 23 | CDR1 | |
| SEQ ID | light chain | SASYRST |
| NO: 24 | CDR2 | |
| SEQ ID | light chain | QHHYSAPWT |
| NO: 25 | CDR3 | |
| SEQ ID | Placeholder | |
| NO: 26 | | |
| SEQ ID | Placeholder | |
| NO: 27 | | |
| SEQ ID | heavy chain | TVSSGGSYIYYADSVKG |
| NO: 28 | CDR2 | |
| SEQ ID | heavy chain | QGTTALATRFFDV |
| NO: 29 | CDR3 | |
| SEQ ID | light chain | KASQDVGTAVA |
| NO: 30 | CDR1 | |
| SEQ ID | light chain | SASYRST |
| NO: 31 | CDR2 | |
| SEQ ID | light chain | QHHYSAPWT |
| NO: 32 | CDR3 | |
| SEQ ID | heavy chain | GFTFSRN |
| NO: 33 | CDR1 | |
| SEQ ID | heavy chain | GFTFSRNGMS |
| NO: 34 | CDR1 | |
| SEQ ID | heavy chain | RNGMS |
| NO: 35 | CDR1 | |
| SEQ ID | heavy chain | SRNGMS |
| NO: 36 | CDR1 | |
| SEQ ID | heavy chain | SSGGSY |
| NO: 37 | CDR2 | |
| SEQ ID | heavy chain | TVSSGGSYIY |
| NO: 38 | CDR2 | |
| SEQ ID | heavy chain | TVSSGGSYIY |
| NO: 39 | CDR2 | |
| SEQ ID | heavy chain | TVSSGGSYIYYADSVKG |
| NO: 40 | CDR2 | |
| SEQ ID | heavy chain | ARQGTTALATRFFDV |
| NO: 41 | CDR3 | |
| SEQ ID | Fc region | EVQLVESGGGLVQPGGSLRLSCAASGFTFSRNGMSWVR |
| NO: 42 | | QAPGKGLEWVATVSSGGSYIYYADSVKGRFTISRDNAKN |
| | | SLYLQMNSLRAEDTAVYYCARQGTTALATRFFDVWGQG |
| | | TLVTVSSASTKGPSVFPLAPSSKSTSGGTAALGCLVKDYF |
| | | PEPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVFLFPPKPKDTLMISRTPEVTCVWDVSHE |
| | | DPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYCTTPPVLDSDGSFFLYSKLTVDKSRWQQGNVFS |
| | | CSVMHEALHNHYTQKSLSCSPGK |
| SEQ ID | Fc region | EVQLVESGGGLVQPGGSLRLSCAASGFTFSRNGMSWVR |
| NO: 43 | | QAPGKGLEWVATVSSGGSYIYYADSVKGRFTISRDNAKN |
| | | SLYLQMNSLRAEDTAVYYCARQGTTALATRFFDVWGQG |
| | | TLVTVSSASTKGPSVFPLAPSSKSTSGGTAALGCLVKDYF |
| | | PEPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNCFS |
| | | CSVMHEALHNHYTQKSLSCSPGK |
| SEQ ID | Fc region | EVQLVrESGGGLVQPGGSLRLSCAASGFTFSRNGMSW\R |
| NO: 44 | | QAPGKGLEWVATVSSGGSYIYYADSVKGRFTISRDNAKN |
| | | SLYLQMNSLRAEDTAVYYCARQGTTALATRFFDVWGQG |
| | | TLVTVSSASTKGPSVFPLAPSSKSTSGGTAALGCLVKDYF |
| | | PEPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSCSPGK |
| SEQ ID | Fc region | EVQLVESGGGLVQPGGSLRLSCAASGFTFSRNGMSWVR |
| NO: 45 | | QAPGKGLEWVATVSSGGSYIYYADSVKGRFTISRDNAKN |
| | | SLYLQMNSLRAEDTAVYYCARQGTTALATRFFDVWGQG |
| | | TLVTVSSASTKGPSVFPLAPSSKSTSGGTAALGCLVKDYF |
| | | PEPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVTLFPPKPKDTLMISRTPEVTCVVVDVSHE |
| | | DPEVKFNWYVDGVEVHNAKTKPREEQYNSTYRWSVLT |
| | | VLHQDWLNGKEYKCKVSNKALPAPIEKTISKAKGQPREP |
| | | CVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSL |
| SEQ ID | Fc region | DIQMTQSPSSLSASVGDRVTITCKASQDVGTAVAWYQQK |
| NO: 46 | | PGKAPKLLIYSASYRSTGVPSRFSGSGSGTDFTLTISSL |
| | | QPEDFATYYCQHHYSAPWTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSCADYEKHKVYACE |
| | | VTHQGLSSPVTKSFNRGEC |
| SEQ ID | Fc region | DIQMTQSPSSLSASVGDRVTITCKASQDVGTAVAWYQQK |
| NO: 47 | | PGKAPKLLIYSASYRSTGVTSRFSGSGSGTDFTLTISSL |
| | | QPEDFATYYCQHHYSAPWTFGGGTKVEIKRTVACPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID | Fc region | TVAAPSVFIFPPSDEQLKSGTASVVCLLNNFYPREAKVQ |
| NO: 48 | | WKVDNALQSGNSQESVTEQDSKDSTYSLSSTLTLSKAAY |
| | | EKHKVYACEVTHQGLSSPVTKSFNRGEC |
| SEQ ID | LCDR1 | TASLSVSSTYLH |
| NO: 49 | | |
| SEQ ID | LCDR2 | STSNLAS |
| NO: 50 | | |
| SEQ ID | LCDR3 | HQYHRSPLT |
| NO: 51 | | |
| SEQ ID | LCDR1 | KASQDVGTAVA |
| NO: 52 | | |
| SEQ ID | LCDR2 | SASYRST |
| NO: 53 | | |
| SEQ ID | LCDR3 | QHHYSAPWT |
| NO: 54 | | |
| SEQ ID | HCDR1 | TKYGVH |
| NO: 55 | | |
| SEQ ID | HCDR2 | VKWAGGSTDYNSALMS |
| NO: 56 | | |
| SEQ ID | HCDR3 | DHRDAMDY |
| NO: 57 | | |
| SEQ ID | HCDR1 | SRNGMS |
| NO: 58 | | |
| SEQ ID | HCDR2 | TVSSGGSYIYYADSVKG |
| NO: 59 | | |
| SEQ ID | HCDR3 | QGTTALATRFFDV |
| NO: 60 | | |
| SEQ ID | IL13Ra2 Ab01 | GCTAGCCCGCCACCATGCTTCTCCTGGTGACAAGCCTT |
| NO: 61 | VLVH scFv | CTGCTCTGTGAGTTACCACACCCAGCATTCCTCCTGAT |
| | | CCCAGACATCCAGATGACCCAGTCCCCCTCTTCTCTGT |
| | | CTGCCTCTGTGGGCGACAGAGTGACCATCACCTGTAAG |
| | | GCCAGTCAGGATGTAGGTACTGCTGTAGCCTGGTATCA |
| | | GCAGAAGCCTGGCAAGGCTCCCAAGCTGCTGATCTAC |
| | | TCGGCATCCTACCGGTCCACTGGCGTGCCTTCCAGATT |
| | | CTCCGGCTCTGGCTCTGGCACCGATTTCACCCTGACCA |
| | | TCTCCTCCCTCCAGCCTGAGGATTTCGCCACCTACTAC |
| | | TGCCAGCACCATTATAGTGCTCCGTGGACGTTTGGCGG |
| | | CGGAACAAAGGTGGAGATCAAGGGTGGTGGTGGTTCT |
| | | GGCGGCGGCGGCTCCGGTGGTGGTGGTTCTGAGGTGC |
| | | AGCTGGTGGAGTCTGGCGGCGGACTGGTGCAGCCTGG |
| | | CGGCTCTCTGAGACTGTCTTGTGCCGCCTCCGGCTTCA |
| | | CCTTCAGTAGGAATGGCATGTCTTGGGTGAGGCAGGC |
| | | CCCTGGCAAGGGCCTGGAGTGGGTGGCCACCGTTAGT |
| | | AGTGGTGGTAGTTACATCTACTATGCAGACAGTGTGAA |
| | | GGGGCGGTTCACCATCTCCAGGGACAACGCCAAGAAC |
| | | TCCCTGTACCTCCAGATGAACTCCCTGAGGGCCGAGGA |
| | | TACCGCCGTGTACTACTGTGCCAGACAAGGGACTACG |
| | | GCACTAGCTACGAGGTTCTTCGATGTCTGGGGCCAGGG |
| | | CACCCTGGTGACCGTGTCCTCTGAATCTAAGTACGGAC |
| | | CG |
| SEQ ID | IL13Ra2 Ab01 | GCTAGCCCGCCACCATGCTTCTCCTGGTGACAAGCCTT |
| NO: 62 | VHVL scFv: | CTGCTCTGTGAGTTACCACACCCAGCATTCCTCCTGAT |
| | Ab01-VL; | CCCAGAGGTGCAGCTGGTGGAGTCTGGCGGCGGACTG |
| | Ab01-VH; | GTGCAGCCTGGCGGCTCTCTGAGACTGTCTTGTGCCGC |
| | ATG start | CTCCGGCTTCACCTTCAGTAGGAATGGCATGTCTTGGG |
| | codon; | TGAGGCAGGCCCCTGGCAAGGGCCTGGAGTGGGTGGC |
| | 5′ NheI | CACCGTTAGTAGTGGTGGTAGTTACATCTACTATGCAG |
| | restriction | ACAGTGTGAAGGGGCGGTTCACCATCTCCAGGGACAA |
| | site (GCTAGC); | CGCCAAGAACTCCCTGTACCTCCAGATGAACTCCCTGA |
| | 3′ RsrII | GGGCCGAGGATACCGCCGTGTACTACTGTGCCAGACA |
| | restriction | AGGGACTACGGCACTAGCTACGAGGTTCTTCGATGTCT |
| | site (CGGACCG) | GGGGCCAGGGCACCCTGGTGACCGTGTCCTCTGGTGGT |
| | | GGTGGTTCTGGCGGCGGCGGCTCCGGTGGTGGTGGTTC |
| | | TGACATCCAGATGACCCAGTCCCCCTCTTCTCTGTCTG |
| | | CCTCTGTGGGCGACAGAGTGACCATCACCTGTAAGGC |
| | | CAGTCAGGATGTAGGTACTGCTGTAGCCTGGTATCAGC |
| | | AGAAGCCTGGCAAGGCTCCCAAGCTGCTGATCTACTC |
| | | GGCATCCTACCGGTCCACTGGCGTGCCTTCCAGATTCT |
| | | CCGGCTCTGGCTCTGGCACCGATTTCACCCTGACCATC |
| | | TCCTCCCTCCAGCCTGAGGATTTCGCCACCTACTACTG |
| | | CCAGCACCATTATAGTGCTCCGTGGACGTTTGGCGGCG |
| | | GAACAAAGGTGGAGATCAAGGAATCTAAGTACGGACC |
| | | G |
| SEQ ID | IL13Ra2 Ab02 | GCTAGCCCGCCACCATGCTTCTCCTGGTGACAAGCCTT |
| NO: 63 | VLVH scFv | CTGCTCTGTGAGTTACCACACCCAGCATTCCTCCTGAT |
| | | CCCAGATATTCAGATGACCCAGAGCCCGAGCAGCCTG |
| | | AGCGCGAGCGTGGGCGATCGCGTGACCATTACCTGCA |
| | | CCGCGAGCCTGAGCGTGAGCAGCACCTATCTGCATTG |
| | | GTATCAGCAGAAACCGGGCAAAGCGCCGAAACTGCTG |
| | | ATTTATAGCACCAGCAACCTGGCGAGCGGCGTGCCGA |
| | | GCCGCTTTAGCGGCAGCGGCAGCGGCACCGATTTTACC |
| | | CTGACCATTAGCAGCCTGCAGCCGGAAGATTTTGCGAC |
| | | CTATTATTGCCATCAGTATCATCGCAGCCCGCTGACCT |
| | | TTGGCGGCGGCACCAAAGTGGAAATTAAAGGTGGTGG |
| | | TGGTTCTGGCGGCGGCGGCTCCGGTGGTGGTGGTTCTG |
| | | AAGTGCAGCTGGTGGAAAGCGGCGGCGGCCTGGTGCA |
| | | GCCGGGCGGCAGCCTGCGCCTGAGCTGCGCGGCGAGC |
| | | GGCTTTACCTTTACCAAATATGGCGTGCATTGGGTGCG |
| | | CCAGGCGCCGGGCAAAGGCCTGGAATGGGTGGCGGTG |
| | | AAATGGGCGGGCGGCAGCACCGATTATAACAGCGCGC |
| | | TGATGAGCCGCTTTACCATTAGCCGCGATAACGCGAA |
| | | AAACAGCCTGTATCTGCAGATGAACAGCCTGCGCGCG |
| | | GAAGATACCGCGGTGTATTATTGCGCGCGCGATCATCG |
| | | CGATGCGATGGATTATTGGGGCCAGGGCACCCTGGTG |
| | | ACCGTGAGCAGCGAATCTAAGTACGGACCG |
| SEQ ID | IL13Ra2 Ab02 | GCTAGCCCGCCACCATGCTTCTCCTGGTGACAAGCCTT |
| NO: 64 | VHVL scFv: | CTGCTCTGTGAGTTACCACACCCAGCATTCCTCCTGAT |
| | Ab02-VL; | CCCAGAAGTGCAGCTGGTGGAAAGCGGCGGCGGCCTG |
| | Ab02-VH; | GTGCAGCCGGGCGGCAGCCTGCGCCTGAGCTGCGCGG |
| | ATG start | CGAGCGGCTTTACCTTTACCAAATATGGCGTGCATTGG |
| | codon; | GTGCGCCAGGCGCCGGGCAAAGGCCTGGAATGGGTGG |
| | 5′ NheI | CGGTGAAATGGGCGGGCGGCAGCACCGATTATAACAG |
| | restriction | CGCGCTGATGAGCCGCTTTACCATTAGCCGCGATAACG |
| | site (GCTAGC); | CGAAAAACAGCCTGTATCTGCAGATGAACAGCCTGCG |
| | 3′ RsrII | CGCGGAAGATACCGCGGTGTATTATTGCGCGCGCGAT |
| | restriction | CATCGCGATGCGATGGATTATTGGGGCCAGGGCACCC |
| | site (CGGACCG) | TGGTGACCGTGAGCAGCGGTGGTGGTGGTTCTGGCGG |
| | | CGGCGGCTCCGGTGGTGGTGGTTCTGATATTCAGATGA |
| | | CCCAGAGCCCGAGCAGCCTGAGCGCGAGCGTGGGCGA |
| | | TCGCGTGACCATTACCTGCACCGCGAGCCTGAGCGTGA |
| | | GCAGCACCTATCTGCATTGGTATCAGCAGAAACCGGG |
| | | CAAAGCGCCGAAACTGCTGATTTATAGCACCAGCAAC |
| | | CTGGCGAGCGGCGTGCCGAGCCGCTTTAGCGGCAGCG |
| | | GCAGCGGCACCGATTTTACCCTGACCATTAGCAGCCTG |
| | | CAGCCGGAAGATTTTGCGACCTATTATTGCCATCAGTA |
| | | TCATCGCAGCCCGCTGACCTTTGGCGGCGGCACCAAA |
| | | GTGGAAATTAAAGAATCTAAGTACGGACCG |
| SEQ ID | Ab01-VH | EVQLVESGGGLVQPGGSLRLSCAASGFTFSRNGMSWVR |
| NO: 65 | | QAPGKGLEWVATVSSGGSYIYYADSVKGRFTISRDNAK |
| | | NSLYLQMNSLRAEDTAVYYCARQGTTALATRFFDVWGQ |
| | | GTLVTVSS |
| SEQ ID | Ab01 HCDR1 | SRNGMS |
| NO: 66 | | |
| SEQ ID | Ab01 HCDR2 | TVSSGGSYIYYADSVKG |
| NO: 67 | | |
| SEQ ID | Ab01 HCDR3 | QGTTALATRFFDV |
| NO: 68 | | |
| SEQ ID | Ab01-VL | DIQMTQSPSSLSASVGDRVTITCKASQDVGTAVAWYQQK |
| NO: 69 | | PGKAPKLLIYSASYRSTGVPSRFSGSGSGTDFTLTISSL |
| | | QPEDFATYYCQHHYSAPWTFGGGTKVEIK |
| SEQ ID | Ab01 LCDR1 | KASQDVGTAVA |
| NO: 70 | | |
| SEQ ID | Ab01 LCDR2 | SASYRST |
| NO: 71 | | |
| SEQ ID | Ab01 LCDR3 | QHHYSAPWT |
| NO: 72 | | |
| SEQ ID | Ab02-VH | EVQLVESGGGLVQPGGSLRLSCAASGFTFTKYGVHWVR |
| NO: 73 | | QAPGKGLEWVAVKWAGGSTDYNSALMSRFTISRDNAK |
| | | NSLYLQMNSLRAEDTAVYYCARDHRDAMDYWGQGTLV |
| | | TVSS |
| SEQ ID | Ab02 HCDR1 | TKYGVH |
| NO: 74 | | |
| SEQ ID | Ab02 HCDR2 | VKWAGGSTDYNSALMS |
| NO: 75 | | |
| SEQ ID | Ab02 HCDR3 | DHRDAMDY |
| NO: 76 | | |
| SEQ ID | Ab02-VL | DIQMTQSPSSLSASVGDRVTITCTASLSVSSTYLHWYQQK |
| NO: 77 | | PGKAPKLLIYSTSNLASGVPSRFSGSGSGTDFTLTISSL |
| | | QPEDFATYYCHQYHRSPLTFGGGTKVEIK |
| SEQ ID | Ab02 LCDR1 | TASLSVSSTYLH |
| NO: 78 | | |
| SEQ ID | Ab02 LCDR2 | STSNLAS |
| NO: 79 | | |
| SEQ ID | Ab02 LCDR3 | HQYHRSPLT |
| NO: 80 | | |
| SEQ ID | DHFRdm | MVGSLNCIVAVSQNMGIGKNGDFPWPPLRNESRYFQRM |
| NO: 81 | | TTTSSVEGKQNLVIMGKKTWFSIPEKNRPLKGRINLVLSR |
| | | ELKEPPQGAHFLSRSLDDALKLTEQPELANKVDMVWIVG |
| | | GSSVYKEAMNHPGHLKLFVTRIMQDFESDTFFPEIDLEKY |
| | | KLLPEYPGVLSDVQEEKGIKYKFEVYEKND |
| SEQ ID | DHFRdm | ATGGTTGGTTCGCTAAACTGCATCGTCGCTGTGTCCCA |
| NO: 82 | | GAACATGGGCATCGGCAAGAACGGGGACTTCCCCTGG |
| | | CCACCGCTCAGGAATGAATCCAGATATTTCCAGAGAA |
| | | TGACCACAACCTCTTCAGTAGAAGGTAAACAGAATCT |
| | | GGTGATTATGGGTAAGAAGACCTGGTTCTCCATTCCTG |
| | | AGAAGAATCGACCTTTAAAGGGTAGAATTAATTTAGTT |
| | | CTCAGCAGAGAACTCAAGGAACCTCCACAAGGAGCTC |
| | | ATTTTCTTTCCAGAAGTCTAGATGATGCCTTAAAACTT |
| | | ACTGAACAACCAGAATTAGCAAATAAAGTAGACATGG |
| | | TCTGGATAGTTGGTGGCAGTTCTGTTTATAAGGAAGCC |
| | | ATGAATCACCCAGGCCATCTTAAACTATTTGTGACAAG |
| | | GATCATGCAAGACTTTGAAAGTGACACGTTTTTTCCAG |
| | | AAATTGATTTGGAGAAATATAAACTTCTGCCAGAATAC |
| | | CCAGGTGTTCTCTCTGATGTCCAGGAGGAGAAAGGCA |
| | | TTAAGTACAAATTTGAAGTATATGAGAAGAATGATTA |
| | | A |
| SEQ ID | GMCSFss | ATGCTTCTCCTGGTGACAAGCCTTCTGCTCTGTGAGTT |
| NO: 83 | Leader | ACCACACCCAGCATTCCTCCTGATCCCA |
| SEQ ID | Ab01-VL | GACATCCAGATGACCCAGTCCCCCTCTTCTCTGTCTGC |
| NO: 84 | | CTCTGTGGGCGACAGAGTGACCATCACCTGTAAGGCC |
| | | AGTCAGGATGTAGGTACTGCTGTAGCCTGGTATCAGCA |
| | | GAAGCCTGGCAAGGCTCCCAAGCTGCTGATCTACTCG |
| | | GCATCCTACCGGTCCACTGGCGTGCCTTCCAGATTCTC |
| | | CGGCTCTGGCTCTGGCACCGATTTCACCCTGACCATCT |
| | | CCTCCCTCCAGCCTGAGGATTTCGCCACCTACTACTGC |
| | | CAGCACCATTATAGTGCTCCGTGGACGTTTGGCGGCGG |
| | | AACAAAGGTGGAGATCAAG |
| SEQ ID | Ab01-VH | GAGGTGCAGCTGGTGGAGTCTGGCGGCGGACTGGTGC |
| NO: 85 | | AGCCTGGCGGCTCTCTGAGACTGTCTTGTGCCGCCTCC |
| | | GGCTTCACCTTCAGTAGGAATGGCATGTCTTGGGTGAG |
| | | GCAGGCCCCTGGCAAGGGCCTGGAGTGGGTGGCCACC |
| | | GTTAGTAGTGGTGGTAGTTACATCTACTATGCAGACAG |
| | | TGTGAAGGGGCGGTTCACCATCTCCAGGGACAACGCC |
| | | AAGAACTCCCTGTACCTCCAGATGAACTCCCTGAGGGC |
| | | CGAGGATACCGCCGTGTACTACTGTGCCAGACAAGGG |
| | | ACTACGGCACTAGCTACGAGGTTCTTCGATGTCTGGGG |
| | | CCAGGGCACCCTGGTGACCGTGTCCTCT |
| SEQ ID | Ab02-VL | GATATTCAGATGACCCAGAGCCCGAGCAGCCTGAGCG |
| NO: 86 | | CGAGCGTGGGCGATCGCGTGACCATTACCTGCACCGC |
| | | GAGCCTGAGCGTGAGCAGCACCTATCTGCATTGGTATC |
| | | AGCAGAAACCGGGCAAAGCGCCGAAACTGCTGATTTA |
| | | TAGCACCAGCAACCTGGCGAGCGGCGTGCCGAGCCGC |
| | | TTTAGCGGCAGCGGCAGCGGCACCGATTTTACCCTGAC |
| | | CATTAGCAGCCTGCAGCCGGAAGATTTTGCGACCTATT |
| | | ATTGCCATCAGTATCATCGCAGCCCGCTGACCTTTGGC |
| | | GGCGGCACCAAAGTGGAAATTAAA |
| SEQ ID | Ab02-VH | GAAGTGCAGCTGGTGGAAAGCGGCGGCGGCCTGGTGC |
| NO: 87 | | AGCCGGGCGGCAGCCTGCGCCTGAGCTGCGCGGCGAG |
| | | CGGCTTTACCTTTACCAAATATGGCGTGCATTGGGTGC |
| | | GCCAGGCGCCGGGCAAAGGCCTGGAATGGGTGGCGGT |
| | | GAAATGGGCGGGCGGCAGCACCGATTATAACAGCGCG |
| | | CTGATGAGCCGCTTTACCATTAGGCGCGATAACGCGAA |
| | | AAACAGCCTGTATCTGCAGATGAACAGCCTGCGCGCG |
| | | GAAGATACCGCGGTGTATTATTGCGCGCGCGATCATCG |
| | | CGATGCGATGGATTATTGGGGCCAGGGCACCCTGGTG |
| | | ACCGTGAGCAGC |
| SEQ ID | (Gly-Gly-Gly- | GGTGGTGGTGGTTCTGGCGGCGGCGGCTCCGGTGGTG |
| NO: 88 | Ser)x3 linker | GTGGTTCT |
| SEQ ID | Ab01 scFv | GACATCCAGATGACCCAGTCCCCCTCTTCTCTGTCTGC |
| NO: 89 | VLVH | CTCTGTGGGCGACAGAGTGACCATCACCTGTAAGGCC |
| | | AGTCAGGATGTAGGTACTGCTGTAGCCTGGTATCAGCA |
| | | GAAGCCTGGCAAGGCTCCCAAGCTGCTGATCTACTCG |
| | | GCATCCTACCGGTCCACTGGCGTGCCTTCCAGATTCTC |
| | | CGGCTCTGGCTCTGGCACCGATTTCACCCTGACCATCT |
| | | CCTCCCTCCAGCCTGAGGATTTCGCCACCTACTACTGC |
| | | CAGCACCATTATAGTGCTCCGTGGACGTTTGGCGGCGG |
| | | AACAAAGGTGGAGATCAAGGGTGGTGGTGGTTCTGGC |
| | | GGCGGCGGCTCCGGTGGTGGTGGTTCTGAGGTGCAGC |
| | | TGGTGGAGTCTGGCGGCGGACTGGTGCAGCCTGGCGG |
| | | CTCTCTGAGACTGTCTTGTGCCGCCTCCGGCTTCACCTT |
| | | CAGTAGGAATGGCATGTCTTGGGTGAGGCAGGCCCCT |
| | | GGCAAGGGCCTGGAGTGGGTGGCCACCGTTAGTAGTG |
| | | GTGGTAGTTACATCTACTATGCAGACAGTGTGAAGGG |
| | | GCGGTTCACCATCTCCAGGGACAACGCCAAGAACTCC |
| | | CTGTACCTCCAGATGAACTCCCTGAGGGCCGAGGATA |
| | | CCGCCGTGTACTACTGTGCCAGACAAGGGACTACGGC |
| | | ACTAGCTACGAGGTTCTTCGATGTCTGGGGCCAGGGCA |
| | | CCCTGGTGACCGTGTCCTCT |
| SEQ ID | Ab01 scFv | GAGGTGCAGCTGGTGGAGTCTGGCGGCGGACTGGTGC |
| NO: 90 | VHVL | AGCCTGGCGGCTCTCTGAGACTGTCTTGTGCCGCCTCC |
| | | GGCTTCACCTTCAGTAGGAATGGCATGTCTTGGGTGAG |
| | | GCAGGCCCCTGGCAAGGGCCTGGAGTGGGTGGCCACC |
| | | GTTAGTAGTGGTGGTAGTTACATCTACTATGCAGACAG |
| | | TGTGAAGGGGCGGTTCACCATCTCCAGGGACAACGCC |
| | | AAGAACTCCCTGTACCTCCAGATGAACTCCCTGAGGGC |
| | | CGAGGATACCGCCGTGTACTACTGTGCCAGACAAGGG |
| | | ACTACGGCACTAGCTACGAGGTTCTTCGATGTCTGGGG |
| | | CCAGGGCACCCTGGTGACCGTGTCCTCTGGTGGTGGTG |
| | | GTTCTGGCGGCGGCGGCTCCGGTGGTGGTGGTTCTGAC |
| | | ATCCAGATGACCCAGTCCCCCTCTTCTCTGTCTGCCTCT |
| | | GTGGGCGACAGAGTGACCATCACCTGTAAGGCCAGTC |
| | | AGGATGTAGGTACTGCTGTAGCCTGGTATCAGCAGAA |
| | | GCCTGGCAAGGCTCCCAAGCTGCTGATCTACTCGGCAT |
| | | CCTACCGGTCCACTGGCGTGCCTTCCAGATTCTCCGGC |
| | | TCTGGCTCTGGCACCGATTTCACCCTGACCATCTCCTC |
| | | CCTCCAGCCTGAGGATTTCGCCACCTACTACTGCCAGC |
| | | ACCATTATAGTGCTCCGTGGACGTTTGGCGGCGGAACA |
| | | AAGGTGGAGATCAAG |
| SEQ ID | Ab02 scFv | GATATTCAGATGACCCAGAGCCCGAGCAGCCTGAGCG |
| NO: 91 | VLVH | CGAGCGTGGGCGATCGCGTGACCATTACCTGCACCGC |
| | | GAGCCTGAGCGTGAGCAGCACCTATCTGCATTGGTATC |
| | | AGCAGAAACCGGGCAAAGCGCCGAAACTGCTGATTTA |
| | | TAGCACCAGCAACCTGGCGAGCGGCGTGCCGAGCCGC |
| | | TTTAGCGGCAGCGGCAGCGGCACCGATTTTACCCTGAC |
| | | CATTAGCAGCCTGCAGCCGGAAGATTTTGCGACCTATT |
| | | ATTGCCATCAGTATCATCGCAGCCCGCTGACCTTTGGC |
| | | GGCGGCACCAAAGTGGAAATTAAAGGTGGTGGTGGTT |
| | | CTGGCGGCGGCGGCTCCGGTGGTGGTGGTTCTGAAGT |
| | | GCAGCTGGTGGAAAGCGGCGGCGGCCTGGTGCAGCCG |
| | | GGCGGCAGCCTGCGCCTGAGCTGCGCGGCGAGCGGCT |
| | | TTACCTTTACCAAATATGGCGTGCATTGGGTGCGCCAG |
| | | GCGCCGGGCAAAGGCCTGGAATGGGTGGCGGTGAAAT |
| | | GGGCGGGCGGCAGCACCGATTATAACAGCGCGCTGAT |
| | | GAGCCGCTTTACCATTAGCCGCGATAACGCGAAAAAC |
| | | AGCCTGTATCTGCAGATGAACAGCCTGCGCGCGGAAG |
| | | ATACCGCGGTGTATTATTGCGCGCGCGATCATCGCGAT |
| | | GCGATGGATTATTGGGGCCAGGGCACCCTGGTGACCG |
| | | TGAGCAGC |
| SEQ ID | Ab02 scFv | GAAGTGCAGCTGGTGGAAAGCGGCGGCGGCCTGGTGC |
| NO: 92 | VHVL | AGCCGGGCGGCAGCCTGCGCCTGAGCTGCGCGGCGAG |
| | | CGGCTTTACCTTTACCAAATATGGCGTGCATTGGGTGC |
| | | GCCAGGCGCCGGGCAAAGGCCTGGAATGGGTGGCGGT |
| | | GAAATGGGCGGGCGGCAGCACCGATTATAACAGCGCG |
| | | CTGATGAGCCGCTTTACCATTAGCCGCGATAACGCGAA |
| | | AAACAGCCTGTATCTGCAGATGAACAGCCTGCGCGCG |
| | | GAAGATACCGCGGTGTATTATTGCGCGCGCGATCATCG |
| | | CGATGCGATGGATTATTGGGGCCAGGGCACCCTGGTG |
| | | ACCGTGAGCAGCGGTGGTGGTGGTTCTGGCGGCGGCG |
| | | GCTCCGGTGGTGGTGGTTCTGATATTCAGATGACCCAG |
| | | AGCCCGAGCAGCCTGAGCGCGAGCGTGGGCGATCGCG |
| | | TGACCATTACCTGCACCGCGAGCCTGAGCGTGAGCAG |
| | | CACCTATCTGCATTGGTATCAGCAGAAACCGGGCAAA |
| | | GCGCCGAAACTGCTGATTTATAGCACCAGCAACCTGG |
| | | CGAGCGGCGTGCCGAGCCGCTTTAGCGGCAGCGGCAG |
| | | CGGCACCGATTTTACCCTGACCATTAGCAGCCTGCAGC |
| | | CGGAAGATTTTGCGACCTATTATTGCCATCAGTATCAT |
| | | CGCAGCCCGCTGACCTTTGGCGGCGGCACCAAAGTGG |
| | | AAATTAAA |
| SEQ ID | S spacer | GAATCTAAGTACGGACCGCCCTGCCCCCCTTGCCCT |
| NO: 93 | | |
| SEQ ID | CD28tm | ATGTTCTGGGTGCTGGTGGTGGTCGGAGGCGTGCTGGC |
| NO: 94 | | CTGCTACAGCCTGCTGGTCACCGTGGCCTTCATCATCT |
| | | TTTGGGTG |
| SEQ ID | 41-BB | AAACGGGGCAGAAAGAAACTCCTGTATATATTCAAAC |
| NO: 95 | | AACCATTTATGAGACCAGTACAAACTACTCAAGAGGA |
| | | AGATGGCTGTAGCTGCCGATTTCCAGAAGAAGAAGAA |
| | | GGAGGATGTGAACTG |
| SEQ ID | CD3ζ | CGGGTGAAGTTCAGCAGAAGCGCCGACGCCCCTGCCT |
| NO: 96 | | ACCAGCAGGGGCAGAATCAGCTGTACAACGAGCTGAA |
| | | CCTGGGCAGAAGGGAAGAGTACGACGTCCTGGATAAG |
| | | CGGAGAGGCCGGGACCCTGAGATGGGCGGCAAGCCTC |
| | | GGCGGAAGAACCCCCAGGAAGGCCTGTATAACGAACT |
| | | GCAGAAAGACAAGATGGCCGAGGCCTACAGCGAGATC |
| | | GGCATGAAGGGCGAGCGGAGGCGGGGCAAGGGCCAC |
| | | GACGGCCTGTATCAGGGCCTGTCCACCGCCACCAAGG |
| | | ATACCTACGACGCCCTGCACATGCAGGCCCTGCCCCCA |
| | | AGG |
| SEQ ID | T2A | CTCGAGGGCGGCGGAGAGGGCAGAGGAAGTCTTCTAA |
| NO: 97 | | CATGCGGTGACGTGGAGGAGAATCCCGGCCCTAGG |
| SEQ ID | EGFRt | CGCAAAGTGTGTAACGGAATAGGTATTGGTGAATTTA |
| NO: 98 | | AAGACTCACTCTCCATAAATGCTACGAATATTAAACAC |
| | | TTCAAAAACTGCACCTCCATCAGTGGCGATCTCCACAT |
| | | CCTGCCGGTGGCATTTAGGGGTGACTCCTTCACACATA |
| | | CTCCTCCTCTGGATCCACAGGAACTGGATATTCTGAAA |
| | | ACCGTAAAGGAAATCACAGGGTTTTTGCTGATTCAGGC |
| | | TTGGCCTGAAAACAGGACGGACCTCCATGCCTTTGAG |
| | | AACCTAGAAATCATACGCGGCAGGACCAAGCAACATG |
| | | GTCAGTTTTCTCTTGCAGTCGTCAGCCTGAACATAACA |
| | | TCCTTGGGATTACGCTCCCTCAAGGAGATAAGTGATGG |
| | | AGATGTGATAATTTCAGGAAACAAAAATTTGTGCTATG |
| | | CAAATACAATAAACTGGAAAAAACTGTTTGGGACCTC |
| | | CGGTCAGAAAACCAAAATTATAAGCAACAGAGGTGAA |
| | | AACAGCTGCAAGGCCACAGGCCAGGTCTGCCATGCCT |
| | | TGTGCTCCCCCGAGGGCTGCTGGGGCCCGGAGCCCAG |
| | | GGACTGCGTCTCTTGCCGGAATGTCAGCCGAGGCAGG |
| | | GAATGCGTGGACAAGTGCAACCTTCTGGAGGGTGAGC |
| | | CAAGGGAGTTTGTGGAGAACTCTGAGTGCATACAGTG |
| | | CCACCCAGAGTGCCTGCCTCAGGCCATGAACATCACCT |
| | | GCACAGGACGGGGACCAGACAACTGTATCCAGTGTGC |
| | | CCACTACATTGACGGCCCCCACTGCGTCAAGACCTGCC |
| | | CGGCAGGAGTCATGGGAGAAAACAACACCCTGGTCTG |
| | | GAAGTACGCAGACGCCGGCCATGTGTGCCACCTGTGC |
| | | CATCCAAACTGCACCTACGGATGCACTGGGCCAGGTCT |
| | | TGAAGGCTGTCCAACGAATGGGCCTAAGATCCCGTCC |
| | | ATCGCCACTGGGATGGTGGGGGCCCTCCTCTTGCTGCT |
| | | GGTGGTGGCCCTGGGGATCGGCCTCTTCATG |
| SEQ ID | DHFRdm | ATGGTTGGTTCGCTAAACTGCATCGTCGCTGTGTCCCA |
| NO: 99 | | GAACATGGGCATCGGCAAGAACGGGGACTTCCCCTGG |
| | | CCACCGCTCAGGAATGAATCCAGATATTTCCAGAGAA |
| | | TGACCACAACCTCTTCAGTAGAAGGTAAACAGAATCT |
| | | GGTGATTATGGGTAAGAAGACCTGGTTCTCCATTCCTG |
| | | AGAAGAATCGACCTTTAAAGGGTAGAATTAATTTAGTT |
| | | CTCAGCAGAGAACTCAAGGAACCTCCACAAGGAGCTC |
| | | ATTTTCTTTCCAGAAGTCTAGATGATGCCTTAAAACTT |
| | | ACTGAACAACCAGAATTAGCAAATAAAGTAGACATGG |
| | | TCTGGATAGTTGGTGGCAGTTCTGTTTATAAGGAAGCC |
| | | ATGAATCACCCAGGCCATCTTAAACTATTTGTGACAAG |
| | | GATCATGCAAGACTTTGAAAGTGACACGTTTTTTCCAG |
| | | AAATTGATTTGGAGAAATATAAACTTCTGCCAGAATAC |
| | | CCAGGTGTTCTCTCTGATGTCCAGGAGGAGAAAGGCA |
| | | TTAAGTACAAATTTGAAGTATATGAGAAGAATGATTA |
| | | A |
| SEQ ID | M spacer | GAATCTAAGTACGGACCGCCCTGCCCCCCTTGCCCTGG |
| NO: 100 | | CCAGCCTAGAGAACCCCAGGTGTACACCCTGCCTCCCA |
| | | GCCAGGAAGAGATGACCAAGAACCAGGTGTCCCTGAC |
| | | CTGCCTGGTCAAAGGCTTCTACCCCAGCGATATCGCCG |
| | | TGGAATGGGAGAGCAACGGCCAGCCCGAGAACAACTA |
| | | CAAGACCACCCCCCCTGTGCTGGACAGCGACGGCAGC |
| | | TTCTTCCTGTACTCCCGGCTGACCGTGGACAAGAGCCG |
| | | GTGGCAGGAAGGCAACGTCTTCAGCTGCAGCGTGATG |
| | | CACGAGGCCCTGCACAACCACTACACCCAGAAGTCCC |
| | | TGAGCCTGAGCCTGGGCAAG |
| SEQ ID | L spacer | ATCTAAGTACGGACCGCCCTGCCCCCCTTGCCCTGCCC |
| NO: 101 | | CCGAGTTCGACGGCGGACCCAGCGTGTTCCTGTTCCCC |
| | | CCCAAGCCCAAGGACACCCTGATGATCAGCCGGACCC |
| | | CCGAGGTGACCTGCGTGGTGGTGGACGTGAGCCAGGA |
| | | AGATCCCGAGGTCCAGTTCAATTGGTACGTGGACGGC |
| | | GTGGAAGTGCACAACGCCAAGACCAAGCCCAGAGAGG |
| | | AACAGTTCCAGAGCACCTACCGGGTGGTGTCTGTGCTG |
| | | ACCGTGCTGCACCAGGACTGGCTGAACGGCAAAGAAT |
| | | ACAAGTGCAAGGTGTCCAACAAGGGCCTGCCCAGCAG |
| | | CATCGAAAAGACCATCAGCAAGGCCAAGGGCCAGCCT |
| | | CGCGAGCCCCAGGTGTACACCCTGCCTCCCTCCCAGGA |
| | | AGAGATGACCAAGAACCAGGTGTCCCTGACCTGCCTG |
| | | GTGAAGGGCTTCTACCCCAGCGACATCGCCGTGGAGT |
| | | GGGAGAGCAACGGCCAGCCTGAGAACAACTACAAGAC |
| | | CACCCCTCCCGTGCTGGACAGCGACGGCAGCTTCTTCC |
| | | TGTACAGCCGGCTGACCGTGGACAAGAGCCGGTGGCA |
| | | GGAAGGCAACGTCTTTAGCTGCAGCGTGATGCACGAG |
| | | GCCCTGCACAACCACTACACCCAGAAGAGCCTGAGCC |
| | | TGTCCCTGGGCAAG |

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through eighth aspects is applicable to all aspects and embodiments identified herein. Moreover, any of the features of an embodiment of the first through eighth aspects is independently combinable, partly or wholly with other embodiments described herein in any way, e.g., one, two, or three or more embodiments may be combinable in whole or in part. Further, any of the features of an embodiment of the first through eighth aspects may be made optional to other aspects or embodiments.

What is claimed is:

1. A nucleic acid encoding a chimeric antigen receptor, the chimeric antigen receptor comprising:
a ligand binding domain that binds to and/or interacts with an IL-13 alpha 2 (IL13Rα2) receptor;
a polypeptide spacer between the ligand binding domain and a transmembrane domain;
the transmembrane domain; and
intracellular signaling region.

2. The nucleic acid of claim 1, wherein the ligand binding domain comprises:
a heavy chain complementarity determining region 1 (CDR1) having the amino acid sequence of SEQ ID NO:20, or conservative variations thereof;
a heavy chain complementarity determining region 2 (CDR2) having the amino acid sequence of SEQ ID NO:21, or conservative variations thereof; and/or
a heavy chain complementarity determining region 3 (CDR3) having the amino acid sequence of SEQ ID NO:22, or conservative variations thereof.

3. The nucleic acid of claim 1 or 2, wherein the ligand binding domain comprises:
a light chain complementarity determining region 1 (CDR1) having the amino acid sequence of SEQ ID NO:23, or conservative variations thereof;
a light chain complementarity determining region 2 (CDR2) having the amino acid sequence of SEQ ID NO:24, or conservative variations thereof; and/or
a light chain complementarity determining region 3 (CDR3) having the amino acid sequence of SEQ ID NO:25, or conservative variations thereof.

4. The nucleic acid of any one of claims 1-3, wherein the ligand binding domain comprises a heavy chain variable (VH) domain comprising a polypeptide having at least 90% identity with the amino acid sequence of SEQ ID NO:18.

5. The nucleic acid of any one of claims 1-4, wherein the ligand binding domain comprises a light chain variable (VL) domain comprising a polypeptide having at least 90% identity with the amino acid sequence of SEQ ID NO:19.

6. The nucleic acid of any one of claims 1-5, wherein the ligand binding domain comprises:
a heavy chain CDR1 having the amino acid sequence of SEQ ID NO:20;
a heavy chain CDR2 having the amino acid sequence of SEQ ID NO:21; and/or
a heavy chain CDR3 having the amino acid sequence of SEQ ID NO:22.

7. The nucleic acid of of any one of claims 1-6, wherein the ligand binding domain comprises:
a light chain CDR1 having the amino acid sequence of SEQ ID NO:23;
a light chain CDR2 having the amino acid sequence of SEQ ID NO:24; and/or
a light chain CDR3 having the amino acid sequence of SEQ ID NO:25.

8. The nucleic acid of any one of claims 1-7, wherein the ligand binding domain comprises a VH domain comprising a polypeptide having the amino acid sequence of SEQ ID NO:18.

9. The nucleic acid of any one of claims 1-8, wherein the ligand binding domain comprises a VL domain having the amino acid sequence of SEQ ID NO:19.

10. The nucleic acid of any one of claims 1-9, wherein the ligand binding domain is an antibody fragment, such as an antigen-binding fragment.

11. The nucleic acid of any one of claims 1-10, wherein the ligand binding domain is a single chain variable fragment (scFv).

12. The nucleic acid of any one of claims 1-11, wherein the scFv comprises a VL-VH orientation.

13. The nucleic acid of any one of claims 1-12, wherein the single chain variable fragment (scFv) comprises a sequence having at least 95% identity with the nucleotide sequence of SEQ ID NO:61.

14. The nucleic acid of any one of claims 1-13, wherein the single chain variable fragment (scFv) comprises the nucleotide sequence of SEQ ID NO:61.

15. The nucleic acid of any one of claims 1-14, wherein the spacer comprises an amino acid sequence of X₁PPX₂P.

16. The nucleic acid of any one of claims 1-15, wherein the spacer region comprises a portion of a hinge region of a human antibody or modified variant thereof.

17. The nucleic acid of any one of claims 1-16, wherein the spacer is 15 amino acids or less but not less than 1 or 2 amino acids.

18. The nucleic acid of any one of claims 1-17, wherein the spacer comprises, consists, or consists essentially of a sequence having at least 95% identity with the amino acid sequence of SEQ ID NO:09.

19. The nucleic acid of any one of claims 1-18, wherein the spacer comprises, consists, or consists essentially of an amino acid sequence of SEQ ID NO:09, or conservative substitutions thereof.

20. The nucleic acid of any one of claims 1-19, wherein the spacer comprises, consists of, or consists essentially of, an IgG4 hinge spacer (S).

21. The nucleic acid of any one of claims 1-20, wherein the spacer comprises, consists of, or consists essentially of, an IgG4 hinge-CH3 spacer (M).

22. The nucleic acid of any one of claims 1-21, wherein the spacer comprises, consists of, or consists essentially of, an IgG4 hinge-CH2 (L234D, N297A)-CHE spacer (L).

23. The nucleic acid of any one of claims 1-22, wherein the intracellular signaling region comprises primary and a costimulatory signaling domains, optionally comprising all or a portion of a CD3 zeta in combination with a co-stimulatory domain selected from the group consisting of signaling domains of CD27, CD28, 4-1BB, OX-40, CD30, CD40, PD-1, ICOS, LFA-1, CD2, CD7, NKG2C, and B7-H3 and combinations thereof.

24. The nucleic acid of claim 23, wherein the intracellular signaling region comprises a signaling portion of a CD3 zeta and a signaling portion of a 4-1BB.

25. The nucleic acid of any one of claims 1-24, wherein the transmembrane domain comprises a CD28 transmembrane domain (CD28tm).

26. The nucleic acid of any one of claims 1-25, further comprising a nucleic acid that encodes a marker sequence.

27. The nucleic acid of claim 26, wherein the marker sequence is a truncated receptor and optionally is an EGFRt, a HER2t, or a CD19t.

28. The nucleic acid of any one of claims 1-27, further comprising a dihydrofolate reductase transgene configured for methotrexate selection.

29. The nucleic acid of claim 28, wherein the dihydrofolate reductase transgene is a dihydrofolate reductase double mutant (DHFRdm).

30. The nucleic acid of claim 29, wherein the dihydrofolate reductase double mutant comprises amino acid mutations of L22F and F31S.

31. An expression vector comprising the nucleic acid of any one of claims 1-30.

32. The expression vector of claim 31, wherein the vector is a viral vector.

33. The expression vector of claim 30 or 31, wherein the vector is a lentiviral or adenoviral vector.

34. A chimeric antigen receptor (CAR) polypeptide encoded by the nucleic acid of any one of claims 1-30.

35. A host cell comprising the nucleic acid of any one of claims 1-30.

36. The host cell of claim 35, wherein the host cell is a CD8+ T cytotoxic lymphocyte cell selected from the group consisting of naïve CD8+ T cells, central memory CD8+ T cells, effector memory CD8+ T cells and bulk CD8+ T cells.

37. The host cell of claim 36, wherein the CD8+ cytotoxic T lymphocyte cell is a central memory T cell and, wherein the central memory T cell is positive for CD45RO+, CD62L+, and CD8+.

38. The host cell of claim 35, wherein the host cell is a CD4+ T helper lymphocyte cell selected from the group consisting of naïve CD4+ T cells, central memory CD4+ T cells, effector memory CD4+ T cells, and bulk CD4+ T cells.

39. The host cell of claim 38, wherein the CD4+ helper lymphocyte cell is a naïve CD4+ T cell and, wherein the naïve CD4+ T cell is positive for CD45RA+, CD62L+ and CD4+ and negative for CD45RO.

40. The host cell of any one of claims 35-39, wherein the host cell is a precursor T cell.

41. The host cell of any one of claims 35-40, wherein the host cell is a hematopoietic stem cell.

42. A pharmaceutical composition comprising the host cell of any one of claims 35-41, and a pharmaceutically acceptable excipient.

43. A method for preparing the host cell of any one of claims 35-41, comprising:
introducing the nucleic acid of any one of claims 1-30 into a cell; and
culturing the cell in the presence of anti-CD3 antibody and/or anti CD28 antibody, and at least one homeostatic cytokine in conditions sufficient for the cells to expand; and
selecting the cell with a selection reagent; wherein the selection reagent is configured to selectively enrich cells transduced with the nucleic acid or vector.

44. A method for preparing a host cell, comprising:
introducing the nucleic acid of any one of claims 1-30 into a cell; and
culturing the cell in the presence of anti-CD3 antibody and/or anti CD28 antibody, and at least one homeostatic cytokine in conditions sufficient for the cells to expand; and
selecting the cell with a selection reagent; wherein the selection reagent is configured to selectively enrich cells transduced with the nucleic acid or vector.

45. The method of claim 43 or 44, wherein the cell is a lymphocyte.

46. The method of claim 45, wherein the lymphocyte has a CD45RA−, CD45RO+, and CD62L+ phenotype.

47. The method of claim 45 or 46, wherein the lymphocyte is CD8+ or CD4+.

48. The method of any one of claims 43-47, wherein the selection reagent is methotrexate.

49. The method of any one of claims 43-48, wherein the cytokine is IL-15, 11-7 and/or Il-21.

50. The method of any one of claims 43-49, further comprising introducing a second nucleic acid into the host cell, wherein the second nucleic acid encodes a marker protein.

51. The method of claim 50, wherein the marker protein is EGFRt.

52. The host cell of any one of claims 35-41 for use in a medicament or for use in the treatment or inhibition of a cancer or a solid tumor expressing an IL-13α2 receptor.

53. The use of claim 52, wherein the cancer comprises a brain cancer.

54. The use of claim 52 or 53, wherein the cancer is a glioma or glioblastoma tumor.

55. The use of claim 54, wherein the cancer is glioblastoma multiforme (GBM).

56. The use of claim 54, wherein the cancer is a glioma.

57. A method of treating, inhibiting, or ameliorating a cancer in a subject, comprising: administering the host cell of any one of claims 35-41 to the subject in need thereof.

58. The method of claim 57, wherein the cancer is a IL13Rα-positive malignancy.

59. The method of claim 57 or 58, wherein the cancer is brain cancer.

60. The method of any one of claims 57-59, wherein the cancer is a glioma or glioblastoma tumor.

61. The method of any one of claims 57-60, wherein the cancer is a glioma.

62. The method of any one of claims 57-60, wherein the cancer is glioblastoma multiforme (GBM).

63. The method of any one of claims 57-62, further comprising administering an additional therapy selected from chemotherapy and radiation therapy.

64. The method of claim 63, wherein the chemotherapeutic drug comprises electochemotherapy, alkylating agent, antimetabolite (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine), anti-tumor antibiotic, topoisomerase inhibitor, mitotic inhibitor, corticosteroid, DNA intercalating agent, or checkpoint inhibitor (checkpoint kinase CHK1, CHK2).

65. The method of any one of claims 57-64, wherein the subject is mammalian.

66. The method of any one of claims 57-65, wherein the subject is human.
